(19) United States
(12) Patent Application Publication
(10) Pub. No.: US 2007/0300324 A1
(43) Pub. Date: Dec. 27, 2007

(54) Method of Producing Sugar and Ethanol from Inflorescence-Deficient Corn Plants

(76) Inventors: Daniel Nadel, Jerusalem (IL); Michael Nadel, Los Angeles, CA (US); Barry Nadel, Kfar Pinnes (IL)

Correspondence Address: JONDLE & ASSOCIATES P.C., 858 HAPPY CANYON ROAD SUITE 230, CASTLE ROCK, CO, 80108, US

(21) Appl. No.: 11/766,551

(22) Filed: Jun. 21, 2007

Related U.S. Application Data
(60) Provisional application No. 60/815,953, filed on Jun. 23, 2006.

(51) Int. Cl. C12N 15/82

(52) U.S. Cl. 800/278

(57) ABSTRACT

Methods for producing high levels of sugars from female-sterile or both female-sterile and male-sterile corn plants are provided. The invention also relates to producing molasses, rum and fodder from the plant material of the sterile corn plants. The invention further relates to using earless corn plants as an economical alternative sugar and ethanol source.

### REFERENCE TO RELATED APPLICATIONS

This application claims priority under 35 U.S.C. §119 to U.S. Application Ser. No. 60/815,953, filed Jun. 23, 2006, which is incorporated herein in its entirety by reference.

### BACKGROUND

The present invention relates to utilizing the culm of corn plants to increase storage of sugars for use in the production of molasses, distilled spirits, refined sugar, ethanol and other products obtained from these corn plants and to methods of using and producing these corn plants. All publications cited in this application are herein incorporated by reference.

Corn is an important crop used as a human food source, animal fodder, silage and as a raw material in industry. The food uses of corn grain, in addition to the human consumption of corn kernels, include products of both the dry milling and wet milling industries. The principal products of dry milling include grits, meal and flour. The principal products of wet milling include starch, syrups and dextrose. “The Economic Feasibility of Ethanol Production from Sugar in the United States”, (2006) The Office of The Chief Economist, USDA.

The industrial applications of corn starch and flour are based on their functional properties, such as viscosity, film formation ability, adhesiveness, absorbent properties and ability to suspend particles. Corn starch and flour are used in the paper and textile industries and as components in adhesives, building materials, foundry binders, laundry starches, diapers, seed treatments, explosives, and oil-well muds. Starch from corn grain or seed is also used extensively in the industry as a source of sugars for producing ethanol. For example, in 2006, 4.86 billion gallons of ethanol were produced in the United States (National Corn Grower's Association, 2006). This was an increase of more than 25 percent over 2005. These numbers show that ethanol is an important fuel which is also increasingly being blended with gasoline.

Accordingly, there remains a need for alternative methods to produce sugar for food and ethanol from corn plants in a more efficient and time-saving manner.

### SUMMARY OF THE INVENTION

The following embodiments and aspects thereof are described in conjunction with systems, tools and methods which are meant to be exemplary, illustrative and not limiting in scope. In various embodiments, one or more of the above-mentioned described problems have been reduced or eliminated, while other embodiments are directed to other improvements.

According to this invention, there is provided a unique method for using female-sterile corn plants or corn plants that are both female-sterile and male-sterile, or corn plants that are inflorescence-deficient for the purpose of increasing sugars stored in the maize culm.

In another aspect, this invention provides for a unique method for producing maize culm comprising high sugar content both 14 days prior to and after anthesis.

In another aspect, this invention provides for producing a female-sterile corn plant or a corn plant that is both female-sterile and male-sterile by introgressing into a maize plant, an allele selected from the group consisting of barren inflorescence1, barren inflorescence2, barren stalk1, barren stalk2, barren stalk3, barren stalk4, barren stalk fastigiate, barren sterile1, defective pistil, lethal ovule2, silkless1, tasselless, teosinte branched1, or other methods of developing inflorescence-deficient maize plants via genetic or transgenetic approaches to engineering male sterility, female sterility, simultaneous male and female sterility, or engineering deficiencies in inflorescence development for the purpose of accumulating sugars in the maize culm.

In another aspect, this invention provides for developing a female-sterile corn plant or a corn plant that is both female-sterile and male-sterile using crossing, selfing, and backcrossing breeding techniques or by transgenic introduction of said corn plants with an allele selected from the group consisting of barren inflorescence1, barren inflorescence2, barren stalk1, barren stalk2, barren stalk3, barren stalk4, barren stalk fastigiate, barren sterile1, defective pistil, lethal ovule2, silkless1, tasselless, teosinte branched1, or by using gametocides, or by cytoplasmic male sterility.

In another aspect, this invention provides for developing inflorescence-deficient maize plants from photoperiodic effects of growing tropical maize in temperate long-day latitudes. Sterility can also be caused by variations in the photoperiod which can induce female sterility, male sterility and/or a combination of female and male sterility by moving short-day tropical corn plants to long-day production zones. Photoperiod-induced inflorescence-deficient corn plants can also include earless and tasselless expression as well as lines that contain tassels and ears which are infertile.

In another aspect, this invention provides for the culm to be used for producing sugar, molasses, distilled spirits, and ethanol from the stalks of female-sterile corn plants that are inflorescence-deficient, or corn plants that are both female-sterile and male-sterile.

In another aspect, this invention provides for the production of heat energy for steam and electricity production from the dried leaves, tassels and residual bagasse from female-sterile corn plants that are inflorescence deficient, or corn plants that are both female-sterile and male-sterile.

In another aspect, this invention provides for the production of fodder having an increased level of sugar and digestibility resulting from inflorescence deficient corn plants.

In another aspect, this invention provides for reduced growing costs of corn plants due to a shortened maturity period for earless corn plants.

In another aspect, this invention provides for an alternative sugar and ethanol source, relative to sweet sorghum, sugarcane and sugar beets, for tropical, subtropical and temperate zones.

In another aspect, this invention provides for multiple plantings and harvests per year using the same plot of land in subtropical, tropical and in some temperate zones through earlier maturity of the corn plant.

In another aspect, this invention provides for growing corn plants in much higher densities than corn plants planted for grain resulting in a reduction in or a lack of, ear formation.

In another aspect, this invention provides for reduction in energy consumption required to convert corn plant biomass into refined sugar, or molasses compared to sugarcane or sugarbeets.

In another aspect, this invention provides for a reduction in fossil fuel use and carbon dioxide emissions generated to produce ethanol or distilled spirits compared to corn grain ethanol production.

In another aspect, this invention provides for a reduction in water utilization per gallon of ethanol produced when compared to corn grain-based ethanol production.

In another aspect, this invention accumulates sucrose and other sugars in maize culm which is substantially less fibrous than sugarcane, thus permitting extraction of juice using less mechanical force and energy than required to extract juice from sugarcane.

In another aspect, this invention enables sequential mechanical harvest and on-site mechanical extraction of sugar juice from the corn plants.

In another aspect, this invention provides for lower cost extraction of high sucrose juice relative to sugarcane or sugar beets due to lack of fiber in the interior of the culm.

In another aspect, this invention provides for less fossil fuel energy and lower carbon dioxide emissions generated to extract sugar juice from the corn plants than either sugarcane or sugar beets.

In another aspect, this invention provides for selection of increased culm or stalk mass as a trait contributing directly to increased juice yields per acre.

In another aspect, this invention provides for a lower harvest cost per kilo of sucrose per acre compared to sugarcane or sugar beets.

In another aspect, this invention provides for a higher yield of sucrose from corn plants as measured by per gram of biomass, and in total biomass per acre than either sugarcane or sugar beets.

In another aspect, this invention provides for higher yield of sugars, and ethanol per acre from corn plants compared to corn grown for grain, sugarcane or sugar beets, or sweet sorghum.

In another aspect, this invention provides for single-stalked and tillerless corn plants at higher plant populations per acre than is possible with sugarcane.

In another aspect, this invention provides for a simultaneous production of a highly digestible fodder crop due to the retention of excess sucrose and hexose sugars within the culm.

In another aspect, this invention enables higher planting density compared to forage corns having ears, allowing higher total yields of green fodder or silage.

In another aspect, this invention provides for use of both earless corn plants and eared corn plants for the production of molasses sucrose, distilled spirits and ethanol production 14 days pre anthesis and 14 days post anthesis.

In another aspect, this invention provides for the elimination of the need to burn plant leaves prior to harvest when compared to sugarcane.

In another aspect, this invention reduces potential habitat for high resident rodent and reptile populations compared to sugarcane fields by drastically reducing time between harvests.

In another aspect, this invention reduces potential habitat for high resident rodent and reptile populations due to more open access to field by avian predators.

In another aspect, this invention provides for lower total carbon dioxide emission prior to harvest when compared to sugarcane as leaves will not be burned off prior to harvest and can be retained to produce energy for processing.

In another aspect this invention provides for lower input growing costs than traditional corn for grain due to early maturity.

In another aspect, this invention allows for a higher production output of ethanol per acre day when compared to sugarcane, sugar beets, sorghum or corn grain.

Further aspects of the invention will become apparent in the description and claims that follow. In addition to the exemplary aspects and embodiments described above, further aspects and embodiments will become apparent by study of the following descriptions.

### DEFINITIONS

In the description and tables that follow, a number of terms are used. In order to provide a clear and consistent understanding of the specification and claims, including the scope to be given such terms, the following definitions are provided:

Allele. An allele is any of one or more alternative forms of a gene, all of which relate to one trait or characteristic. In a diploid cell or organism, the two alleles of a given gene occupy corresponding loci on a pair of homologous chromosomes.

Anthesis. Anthesis means the period or act of flowering.

Backcross. Backcross is the cross of a hybrid to either one of its parents. The offspring of such a cross is referred to as the backcross generation.

Bagasse. Bagasse means the biomass remaining after corn stalks are crushed to extract their juice. Bagasse is often used as a primary fuel source for sugar mills; when burned in quantity, bagasse produces sufficient heat energy to supply all the needs of a typical sugar mill, with energy to spare.

Barren stalk 3-Nadel (ba3-Nadel). Ba3-Nadel is a multi-genic barren stalk trait wherein an earless phenotype is conditioned when plants are grown under long-day photoperiods. (D. Nadel et al 1990.) Ba3-Nadel is distinct and different than the recessive gene barren stalk 3 described by Pan, Y. B. and Peterson, P. A. in “ba3: A new barren stalk mutant in Zea mays L.”, J. Genet. and Breed. 46: 291-294 (1992).

Brix (Bx). Brix (Bx) is a measurement of the mass ratio of dissolved sucrose to water in a liquid. Brix is measured with a saccharimeter that measures specific gravity of a liquid or is measured with a refractometer. A 25° Bx solution has 25 grams of sucrose sugar per 100 grams of liquid. That is, there are 25 grams of sucrose sugar and 75 grams of water in the 100 grams of solution.

Chimeric Gene. A chimeric gene refers to a gene where in nature, the coding sequence is not associated with the endogenous promoter or not associated with at least one other regulatory region of the DNA in the gene.

Corn. Corn, or maize means any inbred, hybrid, progeny or population of Zea mays L. and all of its subspecies.

Culm. The stalk, or stalks of a maize plant excluding the leaves, inflorescences and roots.

Decreased Maturity Date. Decreased maturity date means precocious maturation or earlier than normal maturation.

Earless corn. Earless corn means any inbred, hybrid, progeny or population of Zea mays L. that does not produce ears consisting of husks, silk, seed and cob.

Embryo. The embryo is the rudimentary plant in a seed. The embryo arises from the zygote.

Endosperm. Endosperm means the nutritive tissue formed within the embryo sac in seed plants. It commonly arises following the fertilization of the two primary endosperm nuclei of the embryo sac by the two male sperm. In a diploid organism the endosperm is triploid.

Ethanol. Ethanol means a flammable, colorless chemical compound, also known as ethyl alcohol or grain alcohol. As a biofuel, ethanol is a clean-burning, high-octane fuel that is produced from renewable sources such as corn plants.

Ethanol per acre day. Ethanol per acre day is a means of quantifying total potential ethanol output of a crop per acre divided by the average number of days to harvest. This provides a means to compare different crops for ethanol output per acre day.

Expression cassette. An expression cassette means a transferable region of DNA comprising a chimeric gene which is flanked by one or more restriction or other sites which facilitate precise excision from one DNA locus and insertion into another.

Extraction. Extraction means a method of using roller mills or other devices to extract the sucrose bearing juice from corn stalks.

Female Reproductive Structure. Female reproductive structure means the female gametes and those portions of the plant that are specialized for the production, maturation and viability of female gametes. Female reproductive structures, which can be an ear of maize, or those portions of a plant that produce the carpel and/or the gynoecium (pistil). The carpel of a plant includes but is not limited to, a stigma, style, ovary and cells or tissues that are comprised by the stigma, style and ovary.

Female-Sterile Plant. A female-sterile plant is a plant that is incapable of producing viable seed when pollinated with functional or viable pollen. Such female sterility can be the result of breeding selection and/or the presence of a transgene. A “conditionally female-sterile plant” refers to a plant which under normal growing conditions is female fertile and which can become female-sterile under specific conditions. In the context of the current invention examples of these conditions comprise the exogenous application of a pro-herbicide or other non-phytotoxic substance. In the context of the current invention such a “female-sterile plant” or “conditionally female-sterile plant” remains male fertile and able to produce viable pollen.

Female-Sterile and Male-Sterile Corn Plant. A female-sterile and male-sterile plant means a corn plant that is both female-sterile and male-sterile and is incapable of producing viable seed or viable pollen.

Fermentable sugars. Fermentable sugars means other hexose sugars including but not limited to dextrose, glucose, galactose and fructose.

Fermentation. Fermentation is the anaerobic metabolic breakdown of a nutrient molecule, such as glucose, without net oxidation. Fermentation does not release all the available energy in a molecule; it merely allows glycolysis (a process that yields two ATP per glucose) to continue by replenishing reduced coenzymes. Depending on which organism it is taking place in, fermentation may yield lactate, acetic acid, ethanol, or other reduced metabolites.

Field Corn. Field corn means corn hybrids, varieties or cultivars of corn grown extensively on large acreages within a broad but defined geographic area for the production of grain and/or forage. Most field corn in the United States is also referred to as “dent” corn, whereas field corn produced in Europe and Argentina is more likely to be referred to as “flint” or “flint-dent” corn.

First Juice Press. First juice press means the juice derived from the corn stalks from the first pass through the roller mills.

Fodder. Fodder means the above-ground portion of a corn plant including stalks, leaves and tassels harvested per unit area. Fodder or animal feed, is any foodstuff that is used specifically to feed livestock.

Gene. A gene refers to any DNA sequence comprising several operably linked DNA fragments such as a promoter and a 5′ regulatory region, a coding sequence and an untranslated 3′ region comprising a polyadenylation site.

Grain. Grain means mature corn kernels produced by commercial growers for on-farm use or for sale to customers, in both cases for purposes other than growing or reproducing the species. Typical customers would include livestock feeders, wet or dry millers, or animal feed formulators.

Gross tons per acre. Gross tons per acre means the total weight of all corn plants stalks cut at ground level, including the tassels and leaves, per acre.

Harvesting. Harvesting means the act or process of gathering a crop.

Heterozygous. Heterozygous means a genetic condition existing when different alleles reside at corresponding loci on homologous chromosomes.

Homozygous. Homozygous means a genetic condition existing when identical alleles reside at corresponding loci on homologous chromosomes.

Hybrid. Hybrid means any offspring of a cross between two genetically unlike individuals (Rieger R., A. Michaelis and M. M. Green, 1968, A Glossary of Genetics and Cytogenetics, Springer-Verlag, N.Y.).

Hybrid Corn: Any genotype having two or more parents including the following examples:
(A) a single cross, i.e., a first generation of cross between two inbred lines;
(B) a double cross, i.e., the first generation of a cross between two single crosses;
(C) a three way cross, i.e., the first generation of a cross between a single cross and an inbred line; or
(D) a top cross, i.e., the first generation of a cross between an inbred line and an open pollinated variety, or the first generation of a cross between a single cross and an open pollinated variety.

In the Field. In the field where inflorescence-deficient corn is grown, or adjacent thereto.

Inbred. Inbred means a substantially homozygous individual plant or variety.

Inflorescence Deficient. Inflorescence deficient refers to maize plants lacking in part or completely the male and female inflorescences, commonly known as tassels and ears, respectively.

Inter-planting. Inter-planting refers to a method of planting seeds or plants in a field that ensures adequate cross-pollination of male sterile or conditionally male-sterile plants by the male-fertile plants. This can be achieved either by random mixing of female and male parent seed in different blends (80/20; 90/10; etc) before planting or by planting in specific field patterns whereby different seeds are alternated. When separate harvesting from different plants is required planting in alternating blocks or rows is preferred.

Juice. Juice means the liquid bearing sugar found within the corn stalk.

Kernel. The kernel means the corn caryopsis comprising a mature embryo and endosperm which are products of double fertilization.

Maize. Maize, or corn, means any inbred, hybrid, progeny or population of Zea mays L. and all of its subspecies.

Male Reproductive Structure. Male reproductive structure means the male gametes and those portions of the plant that are specialized for the production, maturation and viability of male gametes. This comprises those portions of a plant that comprise, for example, microspores, stamens, tapetum, anthers and the pollen.

Male-Sterile Plant. Male-sterile plant means a plant that is incapable of supporting viable pollen formation. Such male sterility can be the result of breeding selection or the presence of a transgene. A “conditionally male-sterile plant” refers to a plant which under normal growing conditions is male fertile and which can become male-sterile under specific conditions. For example these conditions might comprise physical emasculation or application of a specific chemical gametocide. In the context of the current invention the said conditions particularly comprise the exogenous application of a pro-herbicide or other non-phytotoxic substance. In the context of the current invention such a “male-sterile plant” or “conditionally male-sterile plant” remains female fertile and able to produce viable seeds when pollinated with functional or viable pollen.

Milk Line. The location where the kernel is harder on the top portion of the kernel and milky on the lower portion of the kernel is the milk line.

Non-Phytotoxic Substances. Non-phytotoxic substances mean substances which are relatively non-phytotoxic to plants, cells or tissues of any particular crop to which the method of the invention is applied. Non-phytotoxic substances need not be non-phytotoxic in all plant tissues of all plants. Non-phytotoxic substances include pro-herbicides. Pro-herbicides are substances with no appreciable direct toxic effect on plant tissues but which are progenitors of active phyto-toxins. In susceptible plant species such pro-herbicides act indirectly as herbicides through the action of endogenous enzymes which convert them in planta to a phyto-toxin.

Net weight. Net weight means the weight in kilograms of the corn plants stalks, excluding the tassels and the leaves.

Percentage dry matter. Percentage dry matter means the total weight of dry matter divided by the total weight of corn stalks, excluding leaves and tassels.

Percentage of sucrose. Percentage of sucrose is the percentage of sucrose present in the juice extracted from corn stalks without the leaves and tassels.

Percentage purity of juice. The percentage purity of juice is the percent of juice extracted from the corn stalks that is not particulate matter from the stalk, dust or other foreign material.

Photoperiod. Photoperiod refers to the relationship between the length of light and dark in a 24 hour period. At the equator, day length is 12 hours of light 12 hours of dark.

Photoperiodic maize. Photoperiodic maize refers to the inflorescence deficiency of maize originating in tropical lowland (below 1200 meters) short-day latitudes that is induced by the longer day length period when the tropical lowland maize is grown in spring and summer of temperate latitudes.

Phyto-Toxins. Phyto-toxins means substances which are toxic to plants, plant tissues and plant cells of the particular crop to which the method of the invention is applied. Such phyto-toxins need not be phyto-toxic to all plant tissues from all plant species.

Plant Density. Plant density means a measurement of the number of corn plants per unit area after planting. As used herein, the plant density is measured in thousands of plants per acre.

Plant Material. Plant material consists of all vegetative matter above the soil.

Promoter Region. Promoter regions refers to a region of DNA comprising at least a functional promoter and, optionally, some or all of its associated upstream regulatory sequences including enhancer sequences and/or associated downstream sequences including some or all of the 5′ untranslated region of the gene endogenous to the promoter.

Residual plant material. Residual plant material means all of the plant material remaining after extraction of the juice.

Second Juice Press. Second juice press is the juice derived from the corn stalks from the second pass through the roller mills.

Seed. The seed is the mature corn kernel produced for the purpose of propagating the species. Alternately, it is a corn kernel commonly sold to commercial grain producers or growers.

Sorghum. Sorghum bicolor (L) Moench and related families of sorghum subspecies is of a genus of numerous species of grasses, some of which are raised for grain and many of which are utilized as fodder plants either cultivated or as part of pasture. Sweet sorghum is also used to produce molasses, ethanol and distilled spirits.

Stalk. The stalk is the stem or culm of a corn plant. As used herein, the stalk is the stem and any residual leaves and tassels attached to the stalk.

Stalk Dry Matter. Stalk dry matter means the net dry bagasse produced after extracting the juice from the corn stalks.

Stalk Yield. Stalk yield means the yield of corn stalk in tons per acre.

Sucrose Yield. Sucrose yield is the amount of sucrose expressed in tons per acre as a percentage of tons of corn stalks harvested, excluding leaves and tassels.

Sugar. Sugar means any of a class of water-soluble crystalline carbohydrates, including but not limited to, sucrose and lactose, having a characteristically sweet taste and classified as monosaccharides, disaccharides, and trisaccharides and also includes any sugar alcohols derived from monosaccharides and disaccharides.

Sugar content. The concentration of sugar per unit area. In corn for example, the sugar content can be measured using Brix readings.

Tassel. An inflorescence of male flowers producing stamens usually located at the apex of the corn plant.

Tasselless corn. Tasselless corn means any inbred, hybrid, progeny or population of Zea mays L. that does not produce a male inflorescence.

Total gross weight of 5 stalks. Total gross weight of 5 stalks means the weight of the corn plant's stalks when cut at ground level and including the tassels and leaves.

Tons of dry matter per acre. Tons of dry matter per acre means the total weight of dry matter of stalks plus the dry matter of leaves and tassels per combined acre in tons.

Tons of stalk per acre. Tons of stalk per acre means the number of tons per acre of corn plant stalks, excluding the leaves and tassels.

Transgenic. Transgenic means any plant in which one or more genes or gene elements has been introduced or altered through the introduction, via physical or agrobacterium-mediated methods, of DNA from another organism, or altered genes or gene elements derived from the host organism itself, or any combination of foreign and host DNA.

Volume Juice % (percentage). Volume juice percentage means the weight of juice extracted, in tons, divided by the number of tons of corn stalks harvested, excluding leaves and tassels.

Wet Leaf Mass. The wet leaf mass means the total quantity of leaves and tassels as measured in tons per acre that is available as fodder.

Wet Mass of Sample. The wet mass of sample means the weight of five stalks of earless corn, including the leaves, tassels, stalk and juice.

### DETAILED DESCRIPTION OF THE INVENTION

This invention is a means of increasing sugar accumulation in a corn plant's culm via inflorescence deficiency without the need for manual or mechanical removal of ears from the stalk.

In typical corn plants that produce ears, studies have shown that the sugar content of the maize stem is reported to decline during the period of rapid kernel growth. Hume, D. J., et al., “Accumulation and translocation of soluble solids in corn stalks” Can. J. Plant Sci. 52:363-368 (1972). Other studies have confirmed that corn kernels serve as the sugar reserves or “sugar sinks” of the corn plant. Felkner, F. C., et al., “Sugar Uptake by Maize Endosperm Suspension Cultures” Plant Physiol. 88:1235-1239 (1988); Chourey, P. S., et al., “Genetic control of cell wall invertases in developing endosperms of maize” Planta 223: 159-167 (2006); Ronginedefekete, M. A., et al. “Mechanism of Glucose Transfer from Sucrose into the Starch Granule of Sweet Corn” Arch Biochem Biophys. 104:173-184 (January 1964); Dickinson, David B. et al., “Presence of ADP-Glucose Pyrophosphorylase in Shrunken-2 and Brittle-2 Mutants of Maize Endosperm” Plant Physiol. 44(7):1058-1062 (July 1969).

It has been shown via manual removal of ears that one of the effects is accumulation of sugars in the culm as reported and confirmed by Christensen, Leslie E, et, al., “The Effects of Ear Removal on Senescence and Metabolism of Maize” Plant Physiol. 68(5):1180-1185 (November 1981); Crafts-Brandner, Steven J., et. al. “Differential Senescence of Maize Hybrids following Ear Removal” Plant Physiol. 74(2):368-373 (February 1984); Singleton, Ralph W, “Segregation for sucrose production in corn stalks”. MNL 23:8-11 (1949); and Shibuya, Tsunetoshi, “Studies of sugar production in corn stalk” MNL 25/29; Singleton, W. R., “Sucrose in the stalks of maize inbreds” Sci. 107:174. (1948).

The present invention encompasses corn plant material having a sugar content of about 6%, 6.3%, 7%, 7.5%, 8%, 8.1%, 9%, 9.6%,10%, 10.5%,11%, 11.2%, 12%, 12.7%, 13%,13.6%, 14%,14.4%,15%,15.9%, 16%,16.7% and 17.0% or higher and including all integers and fractions thereof. The present invention also encompasses corn plant material having a sugar content ranging for example, between 6% to 9%, between 6.5% to 14.4% or between 7.0% to 17.0%, including all integers and fractions thereof.

The present invention also encompasses corn plant material harvested per acre per crop sufficient to produce gallons of ethanol per acre of about 550 gallons, 630 gallons, 710 gallons, 750 gallons, 787 gallons, 800 gallons, 807 gallons, 850 gallons, 863 gallons, 900 gallons, 922 gallons, 950 gallons, 1000 gallons, 1026 gallons, 1050 gallons, 1074 gallons, 1100 gallons, 1133 gallons, 1150 gallons, 1153 gallons, 1200 gallons, 1202 gallons and 1250 gallons of ethanol per acre or higher, and including all integers and fractions thereof. The present invention also encompasses corn plant material harvested per acre per crop sufficient to produce gallons of ethanol per acre ranging for example, between about 550 gallons to 922 gallons, between 630 gallons to 1050 gallons or between 710 gallons to 1250 gallons, including all intergers and fractions thereof.

The present invention also encompasses a corn plant density, the number of plants per acre ranging between any of the following numbers, of 41,000 plants, 42,000 plants, 43,000 plants, 44,000 plants, 45,000 plants, 46,000 plants, 47,000 plants, 48,000 plants, 49,000 plants, 50,000 plants, 51,000 plants, 52,000 plants, 53,000 plants, 54,000 plants, 55,000 plants, 56,000, plants, 57,000 plants, 58,000 plants, 59,000 plants, 60,000 plants, 61,000 plants, 62,000 plants, 63,000 plants, 64,000 plants, 65,000 plants, 66,000 plants, 67,000 plants, 68,000 plants, 69,000 plants, 70,000 plants, 71,000 plants, 72,000 plants, 73,000 plants, 74,000 plants, 75,000 plants, 76,000 plants, 77,000 plants, 78,000 plants, 79,000 plants, 80,000 plants and 81,000 plants, and including all integers and fractions thereof. The present invention also encompasses a corn plant density ranging for example, between 41,000 plants to 55,000 plants, between 42,000 plants to 68,000 plants or between 45,000 plants to 81,000 plants.

Prior to this invention, inflorescence deficient corn plants have not been commercially developed to produce high sugar content. In addition, it was unexpectedly found that in trials of hybrid earless corn plants, brix readings ranging between 10.7% to 20% and sucrose levels ranging between 6.9% to 17.8% in the stalk were obtained.

The present invention encompasses juice derived from corn plant material having a Brix value of about 10.7%,10.9%,11.3%,11.5%,11.8%,12.0%,12.2%,12.4%, 12.6%, 12.9%, 13.1%, 13.5%, 13.6%, 13.8%, 14.2%, 14.3%, 14.5%, 14.7%, 15.1%, 15.2%, 15.4%, 15.8%, 16.0%, 16.2%, 16.5%, 16.8%, 17.3%, 17.5%, 17.8%, 18.1%, 18.3%,18.6%,18.8%,19.0%,19.2%,19.4%,19.7% and 20.0% or higher, and including all intergers and fractions thereof. The present invention also encompasses juice derived from corn plant material having a Brix value ranging for example, between 10.7% to 16.8%, between 10.9% to 19.2% or between 10.7% to 20.0%.

The present invention encompasses sucrose levels in juice of about 6.9%, 7.1%, 7.5%, 7.8%, 8.2%, 8.5%, 8.8%, 9.0%, 9.2%, 9.4%, 9.6%, 9.9%,10.1%,10.4%, 10.7%, 11.0%, 11.3%, 11.5%, 11.8%, 12.0%, 12.1%, 12.5%, 12.6%, 12.8%, 13.1%, 13.3%, 13.6%, 13.9%, 14.2%, 14.4%, 14.7%, 14.8%, 15.0%, 15.3%, 15.4%, 15.7%, 15.9%, 16.1%,16.3%,16.7%,17.0%,17.2%,17.4%,17.8% and 18.0% or higher, and including all integers and fractions thereof. The present invention also encompasses sucrose levels in juice ranging for example, between 6.9% to 17.8%, between 7.1% to 16.7% or between 6.9% to 17.8%.

An important aspect of the present invention is that the present invention can be used to produce ethanol as a biofuel, an important fuel and industrial chemical. Biofuels are an attractive alternative to the commonly used commodity fossil fuels in that they can be both locally produced as well as produced over a widespread area.

Another important advantage of the present invention is that the corn plant material of this invention yields a greater amount of biofuel per acre than conventional corn. For the biomass fuel user, the high sugar content of the present invention provides a substantial two-fold benefit: more sugar may be extracted from any given volume of corn plant material than conventional corn plant material, in turn allowing more ethanol to be extracted from any given volume of corn plant material than the conventional corn plant material.

Another important advantage of the present invention is that it provides a higher economic value in corn to the conventional corn producer. Because the corn plant is not being harvested for the ears and grain, the crop matures substantially earlier than a crop that is harvested for the ears and grain. In the Midwest, Western and Southern states of the United States, this allows for the production of a double crop in one year. In warmer areas such as Brazil, Colombia, Hawaii, Florida or India, this allows for a continuous year-round production of the crop.

Another important advantage of the present invention is that the present invention can be adapted to all current corn production areas.

Another-important advantage of the present invention is that it allows growers to plant a higher density of corn plants than the conventional corn plant grower.

Another important advantage of the present invention is that it allows growers to chose between planting corn for grain or inflorescence deficient corn for sugar, ethanol, molasses, distilled spirits, fodder, silage or any other product derived from this invention depending on existing market conditions.

Another important advantage of the present invention is that the increased level of sugar results in increased digestibility of the silage, or green fodder produced from the corn plants of this invention because it does not contain corn grain and cobs, which are less digestible than sugar.

Another important advantage of the present invention is that when compared to sugarcane, this invention does not require leaf-burning prior to harvesting. This results in a three-fold benefit: 100% of the corn plant material is used either in energy production or silage, CO₂ emissions are reduced and growers can have a dual income from the production of sugar/ethanol and silage from the leaves, tassels and bagasse or from the sale of the leaves, tassels, and bagasse for use as fuel. This assumes that juice is extracted in the field and not sold as cane to local processing plant.

Another important advantage of the present invention is that when compared to sugarcane, less mechanical pressure and time is needed to extract the juice from the corn stalks due to their soft, pithy internal structure.

### Further Embodiments of the Invention

Maize plants (Zea mays L.) present a unique situation in that they can be bred by both self-pollination and cross-pollination techniques. Maize has male flowers, located on the tassel, and female flowers, located on the ear, of the same plant. It can self-pollinate or cross-pollinate. Natural pollination occurs in maize when wind blows pollen from the tassels to the silks that protrude from the tops of the incipient ears.

The reproductive cycle requires that pollen from a male inflorescence pollinate each individual piece of silk that is the receptor of the female inflorescence in order to develop an individual seed on the ear. With full pollination all of the silk will receive pollen and produce an entire ear of corn.

Corn Kernels/Seed as Sugar Sinks

Corn grain or seed is the final product in the reproductive cycle of corn. After the initial vegetative growth achieves sexual maturity male and female inflorescences are produced by the plant. This process requires the transport of sugars from storage in the culm to be used to create the male and female inflorescences and the production of viable pollen and embryos. The reproductive process utilizes a very significant percent of the stored sugar to create seed. C. Y. Tsai, et. al., “Enzymes of Carbohydrate Metabolism in the Developing Endosperm of Maize”; Creech, R. G., “Genetic Control of Carbohydrate Synthesis in Maize Endosperm” Genetics 52:1175-1186 (1965); Makela, P., et. al., “Imaging and Quantifying Carbohydrate Transfer to the Developing Ovaries of Maize” Annals of Botany 96(5):939-949 (2005);

The production of an ear of corn requires many different energy sinks which filter the efficiency of storing carbohydrate energy in the form of seed. Those energy sinks include creation of the male inflorescence (tassel), creation of viable pollen, creation of the female inflorescence (ear), which includes, cob, silk, husk, embryos, fertilized embryos, husks, and shank. Each of these structures requires the use of sugars derived from photosynthesis and stored in the culm for future use in vegetative and reproductive growth. McLaughlin J., et. al., “Glucose Localization in Maize Ovaries When Kernel Number Decreases at Low Potential and Sucrose is Fed to the Stems” Annals of Botany 94(1):75-86 (July 2004); Salvador, R. J., et. al., “Proposed Standar System of Nomenclature for Maize Grain Filling Events and Concepts” Maydica 40(2)141-146.

Each structure created to facilitate the reproductive cycle represents an energy sink that lowers the total carbohydrate pool available to the plant to convert into seed.

Corn Grain as a Source of Sugar, Ethanol, Silage and Other Derived Products

Corn for silage is typically harvested at 60-70% moisture. This is usually determined when the milk line is 50-66% over the way down the immature kernels depending upon the latitude, local microclimates, and varieties of corn used maturity ranges from 95-115 days.

Corn grain requires several steps to be convert into ethanol. It is first converted to starch by wet or dry milling processes, converted from starch to high fructose corn syrup, and the high fructose corn syrup is diluted with water and then fermented. After fermentation it is distilled to produce 96% pure ethanol. The energy for each processing step comes from fossil fuels.

Processing corn grain into ethanol consumes 3-5 gallons of water for each gallon of ethanol produced. Keeney, David, et. al., “Water Use in Ethanol Plants: Potential Challenges, The Institute for Agriculture and Trade Policy” (2006); Moreira, Jose Roberto, “Water Use and Impacts Due Ethanol Production in Brazil” 1 National Reference Center on Biomass, Institute of Electrotechnology and Energy—CENBIO/IEE, University of São Paulo, São Paulo, Brazil. This generates need for waste water treatment, which also is typically powered by fossil fuels.

Corn grain is the largest single crop in the USA with plantings of approximately 90 million acres in 2007. It is widely adapted for day length and can be grown commercially in all states excepting Alaska. It can be successfully cultivated between 0° to 43° latitude, which encompasses a very wide range of prime agricultural land.

Corn grain is a fungible product that can readily be stored for extended periods of time. It is easily transportable in bulk from production areas to storage silos or processing plants. There is very extensive infrastructure in place worldwide for storing and transporting corn grain to the marketplace.

Corn grain and the ingredients produced from it in the milling processes are used in thousands of different products and processes. Industrial uses of seed corn mainly consist of the use of corn starch produced by wet milling of corn grain or seed, and corn flour produced by dry milling of corn grain or seed and whole kernel fermentation for production of food-grade and industrial use ethanol.

Production of corn for grain averaged 147.9 bushels per acre in the USA in 2005. Yields rise and drop with the effect of improved genetics and weather during the growing season.

A bushel of corn is 56 pounds and will produce approximately 2.75-2.90 gallons of ethanol depending upon the processing method. On average, approximately 407-429 gallons of ethanol can be produced from an acre of corn grain produced in the USA. Corn grain takes approximately 160 days to mature, allowing it to produce approximately 2.54-2.68 gallons of ethanol per acre day.

Corn grain is covered by a tough pericarp which protects the seed. The pericarp is indigestible by humans and cattle. Corn which is not adequately dehulled or cracked will pass through a cow's stomach undigested, which limits the caloric value of whole grained corn whether dry or in the form of silage. Silage is not ground fine enough to open the majority of immature seed on the cobs at time of harvest. Hence, a significant percent of the grain passes through the animal and remains intact.

Sugar Beets as a Source of Sugar, Ethanol, Silage and Other Derived Products

Sugar beets (Beta vulgaris L.) are cultivated in the USA as a source of sucrose and molasses and can also be used for the production of ethanol.

Sugar beets can be cultivated from approximately 30-55 degrees latitude.

Sugar beets can be stored for months if necessary, however, due to their cumbersome shape and size they are generally used for sugar production immediately after harvest.

Sucrose is extracted from sugar beets using the diffusion method which, after washing and shredding the sugar beets, adds water to them as a carrying agent. The mixture is centrifuged to extract the sugars present.

US sugar beet production averaged 22.2 tons per acre in 2005 with a recovery of 15.8% sucrose or 3.5 tons per acre. Based upon the US Department of Energy's theoretical Ethanol Yield Calculator, this would produce approximately 606 gallons of ethanol per acre. Sugar beets mature in approximately 220 days allowing the production of approximately 2.75 gallons of ethanol per acre day.

Sugarcane as a Source of Sugar, Ethanol, Silage and Other Derived Products

Sugarcane (Saccharum spp) is cultivated in the USA as a source of sucrose and molasses and can also be used for the production of ethanol.

Sugarcane is cultivated between 0 and 32 degrees latitude with the majority of production located in the tropics below 25 degrees latitude. Subsequently production of sugarcane in the USA is limited to Florida, Louisiana, Texas and Hawaii.

Sugarcane production in the USA in 2005 encompassed 922,000 acres. It averaged 28.8 tons per acre with a recovery of approximately 12.33% sucrose for 3.55 tons per acre. Based upon the US Department of Energy's Theoretical Ethanol Yield Calculator, this would produce approximately 613 gallons of ethanol per acre. Sugarcane matures in approximately 360 days allowing it to produce approximately 1.70 gallons of ethanol per acre day.

Sugarcane requires processing immediately after harvest and possesses sucrose in the juice inside the stalk. The juice is extracted by passing the sugarcane stalks through a three roller mill or first through a shredder and then through a mill. The residual dry stalk material (bagasse) is widely used as a fuel for producing all of the steam and electricity needed to process either finished sugar or ethanol. Neither corn grain nor sugar beets provide any portion of the energy used in their processing. Sugarcane provides a positive net energy and CO₂ balance, which offsets the lower number of gallons per acre day than either sugar beets or corn.

Sugarbeets and sugarcane are principally grown in the USA to produce sucrose. Corn grain is also used to produce sweetners in the form of high fructose corn syrup and dextrose. All three of these sources are widely used by industry and consumers as natural sweetners with carbohydrate content.

Sorghum as a Source of Sugar, Ethanol, Silage and Other Derived Products

Sorghum (Sorghum bicolor (L.) Moench) is a widely adapted grain, fodder, and sweetener crop that can be grown between 0° to 40° latitude. It requires considerably less water in cultivation than corn and can be grown under much more arid conditions.

Sweet sorghum produces both grain and a readily distillable sweet juice in the stalk. It matures in 115-130 days. It is capable of producing sufficient biomass and sugar to produce 400-500 gallons of ethanol per acre. This enables sweet sorghum to produce 3.85-4.35 gallons of ethanol per acre day in the USA.

Sweet sorghum is widely used as a forage crop to produce green fodder or silage. It also produces grain, which can be used for animal feed, human consumption, or for ethanol. Bian YunLong, et al., “QTLs for sugar content of stalk in sweet sorghum (Sorghum bicolor L. Moench)”. Agricultural Sciences in China 5(10):736-744 (2006); Németh, T., “Relationships between nitrogen supply, dry matter accumulation and micro element content of silage sorghum (Sorghum bicolor L./Moench.)” Cereal Research Communications 34(1(II)):593-596 (2006); Reddy, B. V. S., et al., Special issue: “Characterization of ICRISAT-bred Sorghum hybrid parents (set I)”. International Sorghum and Millets Newsletter 47:138 (2006); Liu, RongHou, et al., “Ethanol fermentation of sweet sorghum stalk juice by immobilized yeast” Transactions of the Chinese Society of Agricultural Engineering 21(9):137-140 (2005); Reddy, B. V. S., et al., “Sweet sorghum—a potential alternate raw material for bio-ethanol and bio-energy” International Sorghum and Millets Newsletter 46:79-86 (2005); and Carmi, A., et al., “Effects of irrigation and plant density on yield, composition and in vitro digestibility of a new forage sorghum variety, Tal, at two maturity stages” Animal Feed Science and Technology 131(1/2):121-133 (2006).

A reliable method of controlling fertility in plants provides an opportunity for improved plant breeding techniques. This is especially true for the development of maize hybrids, which typically relies upon some type of male sterility system and sometimes a female sterility system.

Breeding Techniques

Virtually all of the commercial corn produced in the United States is produced from hybrid seed. The production of hybrid seed first requires the development of elite corn inbred lines that possess good combining ability to produce agronomically superior hybrids. The majority of hybrid seed produced in the United States is of the single cross type, wherein two inbred lines are inter-mated, or crossed, to produce what is termed an F₁ single cross hybrid. The resulting kernels from this inter-mating are then sold as seed to commercial growers who plant the seed and harvest the second generation, or F₂ grain, for use on a farm or for commercial sale.

The development of maize hybrids requires the development of homozygous inbred lines, the crossing of these lines, and the evaluation of the crosses. Pedigree breeding and recurrent selection are two of the breeding methods used to develop inbred lines from populations. Breeding programs combine desirable traits from two or more inbred lines or various broad-based sources into breeding pools from which new inbred lines are developed by selfing and selection of desired phenotypes. A hybrid maize variety is the cross of two such inbred lines, each of which may have one or more desirable characteristics lacked by the other or which complement the other. The new inbreds are crossed with other inbred lines and the hybrids from these crosses are evaluated to determine which lines have commercial potential. The hybrid progeny of the first generation is designated F₁. In the development of hybrids only the F₁ hybrid plants are sought. The F₁ hybrid is more vigorous than its inbred parents. This hybrid vigor, or heterosis, can be manifested in many ways, including increased vegetative growth and increased yield.

Anther traditional breeding technique is backcrossing. Backcross breeding has been used to transfer genes for a simply inherited, highly heritable trait into a desirable homozygous cultivar or inbred line which is the recurrent parent. The source of the trait to be transferred is called the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

Backcrossing methods can be used with the present invention to improve or introduce a characteristic into the inbred line. The term “backcrossing” as used herein refers to the repeated crossing of a hybrid progeny back to the recurrent parent, i.e., backcrossing 1, 2, 3, 4, 5, 6, 7, 8 or more times to the recurrent parent. The parental corn plant that contributes the gene for the desired characteristic is termed the nonrecurrent or donor parent. This terminology refers to the fact that the nonrecurrent parent is used one time in the backcross protocol and therefore does not recur. The parental corn plant to which the gene or genes from the nonrecurrent parent are transferred is known as the recurrent parent as it is used for several rounds in the backcrossing protocol (Poehlman & Sleper, 1994; Fehr, 1987). In a typical backcross protocol, the original line of interest (recurrent parent) is crossed to a second line (nonrecurrent parent) that carries the single gene of interest to be transferred. The resulting progeny from this cross are then crossed again to the recurrent parent and the process is repeated until a corn plant is obtained wherein essentially all of the desired morphological and physiological characteristics of the recurrent parent are recovered in the converted plant, in addition to the single transferred gene from the nonrecurrent parent.

Hybrid maize seed can be produced by a male sterility system incorporating manual detasseling. To produce hybrid seed, the male tassel is removed from the growing female inbred parent, which has been planted in alternating rows with the male inbred parent. Consequently, providing that there is sufficient isolation from sources of foreign maize pollen, the ears of the female inbred will be fertilized only with pollen from the male inbred. The resulting seed is therefore a hybrid and will form hybrid plants.

The natural variation in plant development can result in plants tasseling after manual detasseling is completed. Additionally, a detasseler will not completely remove the tassel of the plant. In any event, the female plant will successfully shed pollen and some female plants will be self-pollinated. This will result in seed of the female inbred being harvested along with the hybrid seed which is normally produced.

Alternatively, the female inbred can be mechanically detasseled by machine. Mechanical detasseling is approximately as reliable as hand detasseling, but is faster and less costly. However, most detasseling machines produce more damage to the plants than hand detasseling. Thus, no form of detasseling is presently entirely satisfactory, and a need continues to exist for alternatives which further reduce production costs and eliminate self-pollination in the production of hybrid seed.

A reliable system of genetic male sterility would provide advantages over detasseling. The laborious detasseling process can be avoided by using cytoplasmic male-sterile (CMS) inbreds. Plants of a CMS inbred are male sterile as a result of factors resulting from the cytoplasmic, as opposed to the nuclear, genome. Thus, this characteristic is inherited exclusively through the female parent in maize plants, since only the female provides cytoplasm to the fertilized seed. CMS plants are fertilized with pollen from another inbred that is not male-sterile. Pollen from the second inbred may or may not contribute genes that make the hybrid plants male-fertile. Usually seed from detasseled normal maize and CMS produced seed of the same hybrid must be blended to insure that adequate pollen loads are available for fertilization when the hybrid plants are grown and to insure diversity.

There can be other drawbacks to CMS. One is an historically observed association of a specific variant of CMS with susceptibility to certain crop diseases. This problem has discouraged widespread use of that CMS variant in producing hybrid maize.

Further, it can be appreciated that control of female fertility has advantages. Currently, once the female inbred is rendered male sterile, and the cross pollination has occurred, the male inbred plant is then physically removed since any inbred seed on the plant cannot be sold and should not be released. This adds to the hybrid production an additional expense through the removal process. However, if the male inbred could be rendered female infertile, it would not be necessary to remove the rows of males, and any chance of inbred seed becoming available is reduced. Approximately 20 percent of acreage in producing a hybrid must be devoted to growing the male inbred. Therefore, hybrid seed is being produced on only 80% of the land being utilized for hybrid production.

One type of genetic male sterility is disclosed in U.S. Pat. Nos. 4,654,465 and 4,727,219 to Brar, et al. However, this form of genetic male sterility requires maintenance of multiple mutant genes at separate locations within the genome and requires a complex marker system to track the genes and make use of the system convenient. Patterson also described a genic system of chromosomal translocations which can be effective, but which are complicated. See U.S. Pat. Nos. 3,861,709 and 3,710,511.

Many other attempts have been made to improve on these drawbacks. For example, Fabijanski, et al., developed several methods of causing male sterility in plants (see EPO 89/3010153.8 publication No. 329,308 and PCT application PCT/CA90/00037 published as WO 90/08828). One method includes delivering into the plant a gene encoding a cytotoxic substance associated with a male tissue specific promoter. Another involves an antisense system in which a gene critical to fertility is identified and an antisense to the gene inserted in the plant. Mariani, et al. also shows several cytotoxin encoding gene sequences, along with male tissue specific promoters and mentions an antisense system. See EP 89/401,194. Still other systems use “repressor” genes which inhibit the expression of another gene critical to male sterility. PCT/GB90/00102, published as WO 90/08829.

Another system useful in controlling male sterility makes use of gametocides. Gametocides are not a genetic system, but rather a topical application of chemicals. These chemicals affect cells that are critical to male fertility. The application of these chemicals affects fertility in the plants only for the growing season in which the gametocide is applied (see Carlson, Glenn R., U.S. Pat. No. 4,936,904, which is incorporated herein by reference). Application of the gametocide, timing of the application and genotype specificity often limit the usefulness of the approach.

There has been little previous incentive to control female fertility, and thus little work in this area. One example is the work by De Greef et al described in European patent publication 0412006; U.S. Pat. No. 5,633,441. There, it was noted that female sterility was useful for producing fruit without seeds, enhanced vegetative biomass production and more flower setting within one season. The system set forth there is much like the male sterility system previously employed: the plant is transformed with a female tissue specific promoter linked to a nucleotide sequence which, when expressed, disturbs development of the cell of the flower, seed, or embryo. A selectable marker is also included for selection of transformed cells.

Another example is the work described in U.S. Pat. No. 6,297,426, where a constitutively female sterile plant with inducible fertility was provided. The rationale behind this system was that it would allow male and female inbreds to be grown together in seed production fields, resulting in considerable cost savings in terms of land-use efficiency and in terms of field activities.

Sterility can also be caused by variations in the photoperiod which can induce female sterility, male sterility and a combination of female and male sterility by moving short day length tropical corn to long day production zones.

### EXAMPLES

The following examples further describe the materials and methods used in carrying out the invention and the subsequent results. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### Example 1

### Method of Invention Using Inflorescence-Deficient Stalks of Earless Corn Hybrids to Produce Plants with a High Percentage of Culm Sugars

Tables 1-3 show results of field trial data performed in South Africa in 1990. Data were obtained using hybrid corn CC1 ba3-Nadel having the barren stalk3-Nadel mutation. CC1 was developed from four open-pollinated varieties: two Midwestern dents (Wilson Farm Reid Yellow Dent and Clarage) and two Southern dents (Yellow Tuxpan and Florida Laguna). Seeds of these female-sterile hybrids were planted in close proximity and were grown to produce corn plants. The corn plants were harvested and juice was extracted from the corn plants. Data presented in Tables 1-3 representing three separate field trials planted between Aug. 28, 1990 and Sep. 3, 1990. Each sample number consists of a separate analysis of five corn plants. Row one shows the plant density in thousands of plants per acre, row two shows the total wet mass of plant material obtained in kilograms, row three shows the total wet mass of plant stalk obtained in kilograms, row four shows the total mass of juice obtained in kilograms, row five shows the extrapolated stalk yield in tons per acre, row six shows the pH of the final juice, row seven shows the Brix percentage of the juice, row eight shows the percentage of sucrose obtained after the stalk material was pressed once, row nine shows the percentage of sucrose obtained after the same stalk material was pressed twice, row ten shows the percentage of sucrose in the stalk, row eleven shows the percentage of pure sucrose, row twelve shows the extrapolated yield of dry processed sucrose in tons per acre, row thirteen shows the extrapolated wet leaf mass in tons per acre and row fourteen shows the extrapolated stalk dry matter as a percentage of the total weight of the stalk. The data in Table 1 shows extrapolated stalk yields of 22.3 tons/acre to 27.5 tons/acre obtained with Brix percentage of Juice unexpectedly ranging from 16.0% to 17.2%. Additionally, sucrose yields unexpectedly ranged from 2.4 tons/acre to 3.1 tons/acre.

TABLE 1

| | | CC1 Sample Number | | | | |
|---|---|---|---|---|---|---|
| Characteristic | | 1 | 2 | 3 | 4 | 5 |
| Plant Density | | 81 | 81 | 81 | 81 | 81 |
| Wet Mass | Total (kg) | 3.80 | 3.98 | 4.09 | 4.34 | 4.59 |
| of Sample | Stalk (kg) | 2.75 | 2.95 | 2.96 | 3.20 | 3.39 |
| | Juice (kg) | 0.82 | 0.99 | 1.00 | 1.02 | 1.02 |
| Stalk Yield (tons/acre) | | 22.3 | 23.9 | 23.9 | 25.9 | 27.5 |
| pH | | 4.9 | 5.0 | 5.1 | 5.1 | 5.1 |
| Brix % of Juice | | 16.0 | 17.2 | 16.2 | 16.8 | 17.2 |
| Percentage | Juice | 13.1 | 13.5 | 12.6 | 13.3 | 14.1 |
| of Sucrose | (1^{st} press) | | | | | |
| | Juice | 12.3 | 12.7 | 11.9 | 12.5 | 13.3 |
| | (2^{nd} press) | | | | | |
| | Stalk | 10.5 | 10.8 | 10.1 | 10.6 | 11.3 |

| | | | | | |
|---|---|---|---|---|---|
| Percentage of Juice | 81.9 | 78.6 | 78.0 | 79.2 | 82.2 |
| Purity | | | | | |
| Sucrose Yield (tons/acre) | 2.4 | 2.6 | 2.4 | 2.8 | 3.1 |
| Wet Leaf Mass | 8.5 | 8.5 | 9.3 | 9.3 | 9.7 |
| (tons/acre) | | | | | |
| Stalk Dry Matter | 34.6 | 33.3 | 30.9 | 31.0 | 31.0 |
| (% of stalk) | | | | | |

The data in Table 2 shows stalk yields of 23.1 tons/acre to 33.2 tons/acre obtained with Brix levels unexpectedly ranging from 17.6% to 18.2%. Additionally, sucrose yields unexpectedly ranged from 2.6 tons/acre to 3.8 tons/acre.

TABLE 2

| | | CC1 Sample Number | | | | |
|---|---|---|---|---|---|---|
| Characteristic | | 1 | 2 | 3 | 4 | 5 |
| Plant Density | | 49 | 49 | 81 | 81 | 81 |
| Wet Mass | Total (kg) | 6.59 | 8.45 | 5.55 | 3.78 | 4.52 |
| of Sample | Stalk (kg) | 5.02 | 6.46 | 4.12 | 2.87 | 3.35 |
| | Juice (kg) | 1.65 | 1.86 | 1.23 | 0.97 | 0.86 |
| Stalk Yield (tons/acre) | | 24.3 | 31.6 | 33.2 | 23.1 | 27.1 |
| pH | | 5.3 | 5.2 | 5.1 | 5.1 | 5.1 |
| Brix % of Juice | | 18.2 | 17.8 | 17.6 | 17.6 | 17.6 |
| Percentage | Juice | 14.2 | 14.2 | 14.2 | 13.9 | 14.0 |
| of Sucrose | (1^{st} press) | | | | | |
| | Juice | 13.3 | 13.3 | 13.3 | 13.0 | 13.1 |
| | (2^{nd} press) | | | | | |
| | Stalk | 11.4 | 11.4 | 11.4 | 11.1 | 11.2 |

| | | | | | |
|---|---|---|---|---|---|
| Percentage of Juice | 78.3 | 79.8 | 80.7 | 78.9 | 79.5 |
| Purity | | | | | |
| Sucrose Yield (tons/acre) | 2.8 | 3.6 | 3.8 | 2.6 | 3.0 |
| Wet Leaf Mass | 5.5 | 9.7 | 11.7 | 7.3 | 9.3 |
| (tons/acre) | | | | | |
| Stalk Dry Matter | 32.8 | 29.0 | 30.0 | 33.3 | 33.3 |
| (% of stalk) | | | | | |

The data in Table 3 shows stalk yields of 21.1 tons/acre to 27.1 tons/acre obtained with Brix levels unexpectedly ranging from 17.0% to 17.6%. Additionally, sucrose yields unexpectedly ranged from 2.4 tons/acre to 3.0 tons/acre.

TABLE 3

| | | CC1 Sample Number | | | | |
|---|---|---|---|---|---|---|
| Characteristic | | 1 | 2 | 3 | 4 | 5 |
| Plant Density | | 49 | 49 | 81 | 81 | 81 |
| Wet Mass | Total (kg) | 6.89 | 6.72 | 4.88 | 4.18 | 3.80 |
| of Sample | Stalk (kg) | 5.39 | 5.21 | 3.35 | 3.12 | 2.62 |
| | Juice (kg) | 1.78 | 1.52 | 1.07 | 1.12 | 0.74 |
| Stalk Yield (tons/acre) | | 26.3 | 25.5 | 27.1 | 25.1 | 21.1 |
| pH | | 5.4 | 5.3 | 5.3 | 5.3 | 5.2 |
| Brix % of Juice | | 17.0 | 17.0 | 17.6 | 17.2 | 17.4 |
| Percentage | Juice | 13.4 | 13.5 | 14.0 | 13.7 | 13.9 |
| of Sucrose | (1^{st} press) | | | | | |
| | Juice | 12.6 | 12.7 | 13.1 | 12.9 | 13.0 |
| | (2^{nd} press) | | | | | |
| | Stalk | 10.7 | 10.8 | 11.2 | 11.0 | 11.1 |

| | | | | | |
|---|---|---|---|---|---|
| Percentage of Juice | 78.9 | 79.5 | 79.8 | 79.8 | 79.8 |
| Purity | | | | | |
| Sucrose Yield (tons/acre) | 2.8 | 2.8 | 3.0 | 2.8 | 2.4 |
| Wet Leaf Mass | 7.3 | 7.3 | 7.3 | 7.3 | 7.3 |
| (tons/acre) | | | | | |

### Example 2

### Method of Invention Using ba3-Nadel Corn Plants—Sugar Content in Stalks of Earless Corn Hybrids is Further Increased in Sugary2 Waxy Backgrounds

Table 4 shows results of field trial data performed in South Africa in 1990. Data were obtained using hybrid corn CC1, sweet×waxy (SW/WXY), 3-WAY and waxy line cross (WXY/LC) (SW is the standard sweet corn line, sugary2 (su2); WXY is waxy endosperm; and LC is a line cross between two sister lines of the ba3-Nadel in a dent background), each having the ba3-Nadel mutation. Seeds of these female-sterile hybrids were planted in close proximity and were grown to produce corn plants. The corn plants were harvested and juice was extracted from the corn plants. The date of planting was Sep. 4, 1990. Each sample number consists of a separate plot of corn plants. Row one shows the plant density in hundred thousand plants per acre, row two shows the total wet mass of plant material obtained in kilograms, row three shows the total wet mass of plant stalk obtained in kilograms, row four shows the total mass of juice obtained in kilograms, row five shows the stalk yield in tons per acre, row six shows the pH of the final juice, row seven shows the Brix percentage of the juice, row eight shows the percentage of sucrose obtained after the stalk material was pressed once, row nine shows the percentage of sucrose obtained after the same stalk material was pressed twice, row ten shows the percentage of sucrose in the stalk, row eleven shows the percentage of juice purity, row twelve shows the yield of dry processed sucrose in tons per acre and row thirteen shows the wet leaf mass in tons per acre. Stalk yields of 25.5 tons/acre to 59.6 tons/acre were obtained with Brix levels unexpectedly ranging from 16.4% to 18.8%. Additionally, sucrose yields unexpectedly ranged from 2.8 tons/acre to 5.1 tons/acre with the highest sucrose yields coming from earless plants in a sugary2 waxy background.

TABLE 4

| | | Sample | | | |
|---|---|---|---|---|---|
| Characteristic | | CC1 | SW/WXY | 3-WAY | WXY/LC |
| Plant Density | | 81 | 49 | 65 | 41 |
| Wet Mass | Total (kg) | 3.95 | 10.9 | 6.13 | 9.22 |
| of Sample | Stalk (kg) | 3.13 | 8.91 | 5.11 | 7.30 |
| | Juice (kg) | 0.80 | 2.88 | 1.75 | 2.58 |
| Stalk Yield (tons/acre) | | 25.5 | 43.3 | 33.2 | 59.6 |
| pH | | 5.3 | 5.3 | 5.2 | 5.0 |
| Brix % of Juice | | 18.0 | 18.8 | 17.8 | 16.4 |
| Percentage | Juice | 13.6 | 14.8 | 15.0 | 12.7 |
| of Sucrose | (1^{st} press) | | | | |
| | Juice | 12.7 | 13.7 | 14.0 | 12.0 |
| | (2^{nd} press) | | | | |
| | Stalk | 10.9 | 11.8 | 12.0 | 10.2 |

| | | | | |
|---|---|---|---|---|
| Percentage of Juice | 75.4 | 78.6 | 84.3 | 77.4 |
| Purity | | | | |
| Sucrose Yield (tons/acre) | 2.8 | 5.1 | 4.0 | 3.0 |
| Wet Leaf Mass | 6.5 | 9.7 | 6.5 | 7.7 |
| (tons/acre) | | | | |
| Pol. Scale (10 cm tube) | 26.1 | 28.4 | 28.9 | 24.5 |

### Example 3

### Method of Invention Using ba3-Nadel Corn Plants—Sugar Content in Stalks of Earless Corn Hybrids is Influenced by the Age of the Plant

Tables 5-7 show results of three separate field trials performed in late spring-early summer in Israel between 1987 and 1990. Planting density varied from 52.6 thousand plants/acre (Table 5) to 77.5 thousand plants/acre (Table 7). Two standard sweet corn lines, su2, containing the ba3-Nadel gene were crossed with each other. The resulting hybrids exhibited a 100% earless phenotype. Seeds of these female-sterile hybrids were planted in close proximity and were grown to produce corn plants. The corn plants were harvested and juice was extracted from the corn plants. Each sample consists of a separate analysis of five corn plants. Row one shows the number of days the stalks were cut after planting, row two shows the plant density in thousands of plants per acre, row three shows the total gross weight of five stalks in kilograms, row four shows the extrapolated gross tons of total biomass per acre, row five shows the net weight of the stripped stalk in kilograms, row six shows the extrapolated number of tons of stalk harvested per acre, row seven shows the amount of juice as a percentage of net stalk weight, row eight shows the pH of the juice, row nine shows the Brix percentage of the juice, row ten shows the percentage of purity of the juice, row eleven shows the sucrose percentage of the juice, row twelve shows the extrapolated amount of dry processed sucrose obtained in tons per acre, row thirteen shows the amount of dry matter as a percentage of the total amount stalk and row fourteen shows the extrapolated tons of dry matter of stalk per acre. At a density of 52.6 thousand plants per acre the percent sucrose optimized after 102 days (Table 5).

TABLE 5

| | Sample | | | | | |
|---|---|---|---|---|---|---|
| Characteristic | 1 | 2 | 3 | 4 | 5 | 6 |
| Days Cut After | 85 | 89 | 95 | 102 | 110 | 115 |
| Planting | | | | | | |
| Plant Density | 52.6 | 52.6 | 52.6 | 52.6 | 52.6 | 52.6 |
| Total Gross Weight | 2.3 | 1.9 | 3.1 | 2.4 | 2.0 | 2.0 |
| of 5 stalks (kg) | | | | | | |
| Gross Tons per Acre | 23.9 | 19.5 | 32.8 | 25.3 | 21.1 | 20.5 |
| Net Weight (kg) | 1.7 | 1.4 | 2.4 | 1.9 | 1.6 | 1.5 |
| Tons of Stalk | 17.5 | 14.2 | 25.3 | 20.0 | 16.8 | 24.3 |
| (per acre) | | | | | | |
| Juice % | 59 | 52 | 62 | 57.5 | 41 | 60 |
| pH | 5.2 | 5.2 | 5.1 | 5.5 | 5.4 | 5.2 |
| Brix % of Juice | 11.2 | 12.9 | 17.0 | 17.0 | 18.5 | 18.5 |
| Percentage of Purity | 65.5 | 68.1 | 67.6 | 87.2 | 84.6 | 77.8 |
| of Juice | | | | | | |
| % Sucrose | 7.3 | 8.8 | 11.5 | 15.7 | 15.7 | 14.4 |
| Sucrose Yield | 0.5 | 0.8 | 1.8 | 2.4 | 2.0 | 1.6 |
| (tons/acre) | | | | | | |
| % Dry Matter | 31.3 | 28.8 | 34.8 | 39.6 | 41.1 | 43.6 |
| Tons of Dry Matter | 7.5 | 5.6 | 11.4 | 10.0 | 8.7 | 11.3 |
| Per Acre | | | | | | |

Table 6 shows that as the number of days the stalks were cut after planting increased, the Brix percentage, the percentage of sucrose obtained and the sucrose yield unexpectedly increases up to 110 days.

TABLE 6

| | Sample | | | | | |
|---|---|---|---|---|---|---|
| Characteristic | 1 | 2 | 3 | 4 | 5 | 6 |
| Days Cut After | 85 | 89 | 95 | 102 | 110 | 115 |
| Planting | | | | | | |
| Plant Density | 61.9 | 61.9 | 61.9 | 61.9 | 61.9 | 61.9 |
| Total Gross Weight | 2.3 | 1.9 | 2.4 | 2.6 | 2.2 | 1.5 |
| of 5 stalks (kg) | | | | | | |
| Gross Tons per Acre | 28.2 | 22.9 | 29.4 | 32.2 | 26.6 | 18.5 |
| Net Weight (kg) | 1.7 | 1.3 | 1.8 | 1.9 | 1.9 | 1.3 |
| Tons of Stalk | 21.1 | 16.1 | 22.3 | 23.5 | 22.9 | 15.5 |
| (per acre) | | | | | | |
| Juice % | 69.5 | 57 | 50 | 55 | 56.5 | 45 |
| pH | 5.1 | 5.2 | 5.1 | 5.3 | 5.2 | 5.5 |
| Brix % of Juice | 11.9 | 10.7 | 16 | 17 | 20 | 17.9 |
| Percentage of Purity | 58 | 67.1 | 70.2 | 89.3 | 88.9 | 84.5 |
| of Juice | | | | | | |
| % Sucrose | 6.9 | 7.2 | 11.3 | 15.2 | 17.8 | 15.1 |
| Sucrose Yield | 0.8 | 0.8 | 1.6 | 2.7 | 3.1 | 1.8 |
| (tons/acre) | | | | | | |
| % Dry Matter | 31.1 | 29.4 | 30.7 | 39.5 | 37.8 | 39.3 |
| Tons of Dry Matter | 8.8 | 6.7 | 9.0 | 12.7 | 10.0 | 7.3 |
| Per Acre | | | | | | |

Table 7 below shows results of field trial data performed in late spring-early summer in Israel in 1990. As the number of days the stalks were cut after planting increased, the Brix percentage, the percentage of sucrose obtained and the sucrose yield unexpectedly increased, with maximum percentages and yield being reached at 102 days to 110 days after planting.

TABLE 7

| | Sample | | | | | |
|---|---|---|---|---|---|---|
| Characteristic | 1 | 2 | 3 | 4 | 5 | 6 |
| Days Cut After | 85 | 89 | 95 | 102 | 110 | 115 |
| Planting | | | | | | |
| Plant Density | 77.5 | 77.5 | 77.5 | 77.5 | 77.5 | 77.5 |
| Total Gross Weight | 2.3 | 2.7 | 3.2 | 2.7 | 2.0 | 1.9 |
| of 5 stalks (kg) | | | | | | |
| Gross Tons per Acre | 35.5 | 41.1 | 47.6 | 41.1 | 30.2 | 28.7 |
| Net Weight (kg) | 1.7 | 2.1 | 2.5 | 2.0 | 1.7 | 1.5 |
| Tons of Stalk | 26.4 | 30.6 | 38.7 | 31.0 | 26.4 | 23.2 |
| (per acre) | | | | | | |
| Juice % | 69.5 | 62.5 | 71.2 | 69.5 | 51.0 | 54.0 |
| pH | 5.2 | 5.2 | 5.3 | 5.3 | 5.3 | 5.4 |
| Brix % of Juice | 11.7 | 13.5 | 15.0 | 17.0 | 20.0 | 18.1 |
| Percentage of Purity | 64.4 | 77.0 | 72.4 | 88.3 | 79.1 | 73.8 |
| of Juice | | | | | | |
| % Sucrose | 7.53 | 10.4 | 10.8 | 15.1 | 15.9 | 13.4 |
| Sucrose Yield | 1.2 | 2.3 | 2.8 | 3.4 | 3.0 | 2.1 |
| (tons/acre) | | | | | | |
| % Dry Matter | 31.7 | 28.1 | 31.0 | 36.6 | 39.7 | 37.8 |
| Tons of Dry Matter | 11.3 | 11.5 | 14.8 | 15.0 | 12.0 | 10.9 |
| Per Acre | | | | | | |

### Example 4

### Stalks of Eared Corn Hybrids Contain a Low Percentage of Sugar

Table 8 shows results of field data performed in late spring-early summer in Israel in 1990. Two standard sweet corn lines, su2, were crossed resulting in normal corn plant phenotypes that produced ears. Seeds of these hybrids were planted in close proximity and were grown to produce corn plants. The corn plants were harvested and juice was extracted from the corn plants. Each sample number consists of a separate plot of five corn plants. Row one shows the number of days the stalks were cut after planting, row two shows the plant density in hundred thousand plants per acre, row three shows the total gross weight of five stalks in kilograms, row four shows the extrapolated gross tons of total biomass per acre, row five shows the net weight stripped stalk in kilograms, row six shows the extrapolated tons of stalk harvested per acre, row seven shows the amount of juice as a percentage of net stalk weight, row eight shows the pH of the juice, row nine shows the Brix percentage of the juice, row ten shows the percentage of purity of the juice, row eleven shows the sucrose percentage of the juice, row twelve shows the extrapolated amount of dry processed sucrose obtained in tons per acre, row thirteen shows the amount of dry matter as a percentage of the total amount of stalk and row fourteen shows the tons of dry matter of stalk per acre. Brix percentages range from 5.1 to 6.8 and the sucrose yields range from 0.3 to 0.5 tons per acre. Sucrose yields peaked at 102 days after planting, but yields were one-sixth of those obtained with earless plants (see Table 7).

TABLE 8

| | Sample | | | | | |
|---|---|---|---|---|---|---|
| Characteristic | 1 | 2 | 3 | 4 | 5 | 6 |
| Days Cut After | 85 | 89 | 95 | 102 | 110 | 115 |
| Planting | | | | | | |
| Plant Density | 77.5 | 77.5 | 77.5 | 77.5 | 77.5 | 77.5 |
| Total Gross Weight | 1.7 | 1.9 | 2.0 | 2.1 | 1.7 | 1.6 |
| of 5 stalks (kg) | | | | | | |
| Gross Tons per Acre | 26.4 | 28.7 | 31.0 | 32.6 | 26.4 | 24.0 |
| Net Weight (kg) | 1.0 | 1.1 | 1.2 | 1.2 | 1.0 | 0.8 |
| Tons of Stalk | 15.5 | 16.2 | 18.6 | 18.6 | 14.7 | 11.6 |
| (per acre) | | | | | | |
| Volume Juice % | 62.5 | 64.5 | 67.0 | 58.0 | 48.5 | 49.0 |
| pH | 5.1 | 5.2 | 5.2 | 5.3 | 5.2 | 5.4 |
| Brix % of Juice | 5.1 | 5.4 | 6.1 | 6.5 | 6.8 | 6.2 |
| Percentage of Purity | 62.2 | 64.3 | 72.2 | 77.3 | 74.3 | 72.1 |
| of Juice | | | | | | |
| % Sucrose | 3.2 | 3.5 | 3.2 | 4.4 | 5.1 | 4.5 |
| Sucrose Yield | 0.3 | 0.4 | 0.4 | 0.5 | 0.4 | 0.3 |
| (tons/acre) | | | | | | |
| % Dry Matter | 33.2 | 34.2 | 35.6 | 39.7 | 41.2 | 43.1 |
| Tons of Dry Matter | 8.7 | 9.8 | 11.1 | 12.9 | 10.9 | 10.4 |
| Per Acre | | | | | | |

### Example 5

### Stalks of Eared Corn Hybrids Contain a Lower Percentage of Sugar than Stalks of Earless Corn Hybrids

Table 9 below shows side-by-side of data extrapolated from tables 7 and 8. Row one shows the number of days the stalks were cut after planting, row two shows the column headers for earless corn plants, designated as “EL”, and eared corn plants, designated as “E”, row three shows the gross tons of total biomass per acre, row four shows the gross tons harvested per acre, row five nine shows the tons of can harvested per acre, row six shows the Brix percentage of the juice, row seven shows the sucrose percentage of the juice and row eight shows the amount of dry processed sucrose obtained in tons per acre. Note the significant differences between the earless corn plants and the eared corn plants of each characteristic at each date. The total gross weight, the gross tons per acre, the tons of stalk harvested per acre, the Brix percentage of the juice, the percentage of sucrose of the juice and the sucrose yield of the earless plants is consistently greater than the eared plants. The Brix percentages of the earless corn plants unexpectedly ranges from between 2.3 to 2.9 times greater than the eared corn plants. Additionally, the sucrose yield of the earless plants unexpectedly ranges from between 4 to 7.5 times greater than the eared corn plants.

TABLE 9

| | 85 | | 89 | | 95 | | 102 | | 110 | | 115 | |
|---|---|---|---|---|---|---|---|---|---|---|---|---|
| Characteristic | EL | E | EL | E | EL | E | EL | E | EL | E | EL | E |
| Total Gross | 2.3 | 1.7 | 2.7 | 1.9 | 3.2 | 2.0 | 2.7 | 2.1 | 2.0 | 1.7 | 1.9 | 1.6 |
| Weight of 5 | | | | | | | | | | | | |
| stalks (kg) | | | | | | | | | | | | |
| Gross Tons | 35.5 | 26.4 | 41.1 | 28.7 | 47.6 | 31.0 | 41.1 | 32.6 | 30.2 | 26.4 | 28.7 | 24.0 |
| per Acre | | | | | | | | | | | | |
| Tons of Stalk | 26.4 | 15.5 | 30.6 | 16.2 | 38.7 | 18.6 | 31.0 | 18.6 | 26.4 | 14.7 | 23.2 | 11.6 |
| (per acre) | | | | | | | | | | | | |
| Brix % of Juice | 11.7 | 5.1 | 13.5 | 5.4 | 15.0 | 6.1 | 17.0 | 6.5 | 20.0 | 6.8 | 18.1 | 6.2 |
| % Sucrose | 7.53 | 3.2 | 10.4 | 3.5 | 10.8 | 3.2 | 15.1 | 4.4 | 15.9 | 5.1 | 13.4 | 4.5 |
| Sucrose Yield | 1.2 | 0.3 | 2.3 | 0.4 | 2.8 | 0.4 | 3.4 | 0.5 | 3.0 | 0.4 | 2.1 | 0.3 |
| (tons/acre) | | | | | | | | | | | | |

### Example 6

### Use of the Barren Stalk1 (ba1) Allele in the Present Invention

Another method of the present invention is corn seeds having the capacity, either inherent or transgenic, to manifest a ba1 mutant phenotype, which results in corn plants having both female and male sterility and planting the corn seeds in close proximity to each other. The seeds are then grown to produce female sterile corn plants having the barren1 mutant phenotype. Plant material is harvested from said plants having the barren1 phenotype and then the juice is extracted from said corn plants.

### Example 7

### Use of the Barren Stalk2 Allele (ba2) in the Present Invention

Another method of the present invention is corn seeds having the capacity, either inherent or transgenic, to manifest a ba2 mutant phenotype, which results in female sterile corn plants and planting the corn seeds in close proximity to each other. The seeds are then grown to produce female sterile corn plants manifesting the ba2 phenotype. Plant material is harvested from corn plants having said ba2 phenotype and then the juice is extracted from said corn plants.

The genetic linkage of ba2 to seed marker miniature seed1 (mn1) for the purpose of identifying among the F₁ hybrid seed those seeds that will produce earless corn plants, represents an extension of this invention. Said seed can be mechanically selected by seed size and/or opacity of seed. Based upon the genetic linkage of mn1 and ba2 approximately 1-2% of said selective seeds will yield plants with ears. Under the planting densities for this invention this small percentage of eared plants among ba2 earless plants will have inconsequential effects on total per acre sucrose yields. The use of barren stalk 3 (ba3) or barren stalk 4 (ba4) in a manner similar to the above described use of ba2 is claimed as an extension of this invention.

### Example 8

### Use of the Barren-Stalk Fastigiate Allele in the Present Invention

Another method of the present invention is corn seeds having the capacity, either inherent or transgenic, to manifest a barren-stalk fastigiated mutant phenotype which results in female sterile corn plants and planting the corn seeds in close proximity to each other. The seeds are then grown to produce female sterile corn plants manifesting the barren-stalk fastigiate phenotype. Plant material is harvested from corn plants having said barren-stalk fastigiate phenotype and then the juice is extracted from said corn plants.

### Example 9

### Use of the Defective Pistil Mutant Allele in the Present Invention

Another method of the present invention is corn seeds having the capacity, either inherent or transgenic, to manifest a defective pistil mutant phenotype, which results in female sterile corn plants and planting the corn seeds in close proximity to each other. The seeds are then grown to produce female sterile corn plants manifesting the defective pistil mutant phenotype. Plant material is harvested from corn plants having said defective pistil mutant phenotype and then the juice is extracted from said corn plants.

### Example 10

### Use of the Lethal Ovule2 Allele in the Present Invention

Another method of the present invention is corn seeds having the capacity, either inherent or transgenic, to manifest a lethal ovule2 phenotype, which results in female sterile corn plants and planting the corn seeds in close proximity to each other. The seeds are then grown to produce female sterile corn plants manifesting the lethal ovule2 phenotype. Plant material is harvested from corn plants having said lethal ovule2 phenotype and then the juice is extracted from said corn plants.

### Example 11

### Use of the Silkless Allele in the Present Invention

Another method of the present invention is corn seeds having the capacity, either inherent or transgenic, to manifest a silkless phenotype, which results in female sterile corn plants and planting the corn seeds in close proximity to each other. The seeds are then grown to produce female sterile corn plants manifesting the silkless phenotype. Plant material is harvested from corn plants having said silkless phenotype and then the juice is extracted from said corn plants.

### Example 12

### Use of the Teosinte Branched1 Allele in the Present Invention

Another method of the present invention is corn seeds having the capacity, either inherent or transgenic, to manifest a teosinte branched1 phenotype, which results in female sterile corn plants, and planting the corn seeds in close proximity to each other. The seeds are then grown to produce female sterile corn plants manifesting the teosinte branched1 phenotype. Plant material is harvested from corn plants having said teosinte branched1 phenotype and then the juice is extracted from said corn plants.

### Example 13

### Use of the Dam Allele in the Present Invention

Another method of the present invention is corn seeds having the allele dam, which results in male sterile corn plants and also having any of the alleles resulting in female sterility so that said corn seeds will result in corn plants that are both male and female sterile. Said corn seeds having dam, and having any of the female sterile alleles, are planted in close proximity to each other. The seeds are then grown to produce corn plants which are both male and female sterile. Plant material is harvested from corn plants having dam, and which are both male and female sterile, and then the juice is extracted from said corn plants.

### Example 14

### Use of the Barnase Allele in the Present Invention

Another method of the present invention is corn seeds having the allele barnase, which results in male sterile corn plants, and also having any of the alleles resulting in female sterility so that said corn seeds will result in corn plants that are both male and female sterile. Said corn seeds having barnase, and having any of the female sterile alleles, are planted in close proximity to each other. The seeds are then grown to produce corn plants which are both male and female sterile. Plant material is harvested from corn plants having barnase, and which are both male and female sterile, and then the juice is extracted from said corn plants.

### Example 15

### Use of the Barstar Allele in the Present Invention

Another method of the present invention is corn seeds having the allele barstar, which results in male sterile corn plants, and also having any of the alleles resulting in female sterility so that said corn seeds will result in corn plants that are both male and female sterile. Said corn seeds having barstar, and having any of the female sterile alleles, are planted in close proximity to each other. The seeds are then grown to produce corn plants which are both male and female sterile. Plant material is harvested from corn plants having barstar, and which are both male and female sterile, and then the juice is extracted from said corn plants.

### Example 16

### Method of Extracting Sugar from Corn Plants Using the Juicing Method

With the present invention sugar production from maize is focused on the production of sucrose from corn stalks. The corn stalks may include some residual pieces of leaves and tassels. Corn stalks of this invention can be harvested using mechanical harvesters. After cutting, the corn stalks can be placed in a large pile and, prior to milling, cleaned. There are typically two steps to the milling process, breaking of the corn stalks and grinding of the corn stalks. Breaking of the corn stalks revolve around using knives, shredders, crushers or a combination of these processes. The grinding of the corn stalks involves using roller mills in multiple sets of three, four, five or more rollers and using conveyers to transport the crushed corn plant material from one mill to another, where it is simultaneously imbibed with water to enhance juice extraction at the next mill. The juice from the mills can then be strained to remove large particles. Next, the juice is clarified using heat (to about 200° F.) and lime (to neutralize any organic acids) and sometimes, small quantities of soluble phosphate. A heavy precipitate forms which is then separated from the juice in the clarifier. The insoluble particulate mass is called “mud” and is separated from the limed juice by gravity or centrifuge. The clarified juice contains the sugar of interest. “Cane Sugar Refining,” Cane Sugar Handbook, pp 435-499, Chen, J. P and Chow, C. C., John Wiley & Sons, Inc. (1993).

### Example 17

### Method of Extracting Sugar from Corn Plants Using the Diffusion Method

In the present invention sugar production from maize is focused on the production of sucrose from corn stalks. The corn stalks may include some residual pieces of leaves and tassels. Corn stalks of this invention are harvested using mechanical harvesters. The corn stalks are cut using knives, shredders, crushers or a combination of these processes. After cutting, conveyor belts send the corn stalks through a diffuser or extractor, where hot water is mixed in with the corn stalks to dissolve and remove the sugar. The corn stalk pulp is squeezed, where the water and sugar juice are saved and the dry pulp is removed. The water and sugar juice is then treated with milk of lime which is treated with carbon dioxide twice and then filtered to remove other non-sugars. The clarified and purified juice contains the sugar(s) of interest. “Cane Sugar Refining,” Cane Sugar Handbook, pp 435-499, Chen, J. P and Chow, C. C., John Wiley & Sons, Inc. (1993)

### Example 18

### Method of Extracting Ethanol from Corn Plants

There are several steps involved in the process of producing ethanol from the juice extracted from the corn stalks, where the corn stalks may include residual pieces of leaves and tassels. Ethanol was produced through a biochemical processes based on fermentation using the juice or molasses as a feedstock (or a mixture of the juice and molasses). The sugars were transformed into alcohol using yeasts as the catalyst. Fermentation can take anywhere from four to twelve hours and liberate a significant amount of carbon dioxide and heat. The fermentation process can be conducted in batch or continuously, using open or closed fermentation tanks to produce ethanol. The resulting ethanol can then be distilled from other by-products in order to achieve any level of purity. Stockholm Environment Institute, Sugarcane Resources for Sustainable Development: A Case Study in Luena, Zambia, April 2001.

For additional methods of producing ethanol from sugar containing substrates, please see U.S. Pat. Nos. 4,326,036, 4,560,659, 4,738,930, 4,876,196 and U.S. Pub. No. 20030143704, all of which are herein incorporated by reference.

### Example 19

### Use of Traditional Breeding Techniques in the Present Invention

This invention also is directed to methods for producing a corn plant by crossing a first parent corn plant with a second parent corn plant wherein the first or second parent corn plant is a corn plant having any of the above-mentioned genes and/or traits. Further, both first and second parent corn plants can have any of the above-mentioned genes and/or traits. Thus, any such methods using corn plants having any of the above-mentioned genes and/or traits for the purpose of isolating and accumulating sugar containing juice or fluids from the culms of said plants are part of this invention: parent lines, selfs, backcrosses, hybrids, crosses to populations, and the like. All plants produced using corn plants having any of the above-mentioned genes and/or traits are within the scope of this invention, including those developed from lines derived from corn plants having any of the above-mentioned genes and/or traits. Advantageously, this corn inbred line could be used in crosses with other, different, corn plants to produce the first generation (F₁) corn hybrid seeds and plants with the above-mentioned characteristics. Genetic variants created either through traditional breeding methods using corn plants having any of the above-mentioned genes and/or traits or through transformation by any of a number of protocols known to those of skill in the art are intended to be within the scope of this invention.

Generation of Corn Plants Rendered Female-Sterile, Male Sterile or both Female-Sterile and Male-Sterile

### Example 20

### Examples of Female Fertility Genes Used in the Present Invention

A female fertility gene may be selected from those genes already known. One such female fertility gene is the teosinte branched1 (tb1) gene, which increases apical dominance, resulting in multiple tassels and repression of female tissue. Doebley, J., Stec., A. and Hubbard, L. “The evolution of apical dominance in maize” Nature 386:485-488 (1977), which, as with all other references cited, is incorporated herein by reference. Another example are the so-called “barren genes”. “barren stalk1” and “barren stalk2” or “ba1” and “ba2” were first described by Hofmeyr, J. D. J. (1931). Barren stalk (ba1) is a spontaneous mutation identified in 1928 from material collected by R. A. Emerson in South America. Hofmeyr, J. D. J. Thesis, Cornell University, Ithaca, N.Y., 1931. Homozygous mutants are unable to produce vegetative branches (tillers), female inflorescences (ears) and a normal apical male inflorescence, the tassel. Ritter, et al., Am. J. Bot. 89: 203-210 (2002). The tassel of ba1 mutants is unbranched, shortened and predominantly sterile, owing to the often complete lack of spikelets, the short branches that represent the basic unit of grass inflorescences. See also Ritter, M. K., et al., “The Maize Mutant Barren Stalk1 is Defective in Axillary Stem Development” Amer. J. of Botany 89:203-210 (2002) and Gallavotti, A., et al., “The role of the barren stalk1 in the architecture of maize” Nature 432: 630-635 (Dec. 2, 2004). Ba2 mutants are characterized by the often absence of ear shoots, the culm may be distorted or cracked, the tassel branches are erect and is fastigiated with branched peduncles fused to the base. There are two types of “barren 3” or “ba3” mutants. The first type is multigenic and was discovered by D. Nadel, et al., “A new 100% earless recessive trait” Maize Genetic Cooperative Newsletter, Vol. 6 (1990). The second mutant was isolated from a mutant maize plant infected with wheat-streak mosaic virus and is described at Pan, Y. B. and Peterson, P. A., “ba3: A new barren stalk mutant in Zea mays L.”, J. Genet and Breed. 46: 291-294 (1992). The plants develop normal tassels but do not have any ear shoots along the stalks. This locus has been identified genetically but not yet cloned and sequenced. Barren-stalk fastigiate, is described in Coe, E. H. and Beckett, J. S. “Barren-stalk fatigiate, baf, chromosome 9S:, Maize Genet. Coop. Newsletter 61:46-47 (1987). Other examples include the Lethal ovule mutant (Vollbrecht, E. “Lethal ovule2 cases aberrant embryo sac development” Maize Genet Coop. Newsletter (No. 68) p. 2-3 (1994)); and defective pistil mutant (Miku V. E. and Mustyatsa S. I. “Phenotypic description and genetic analysis of a mutation causing female sterility in corn” Genetika 14(2): 365-368 (1978).

Inducible Promoters Used in the Present Invention

In the practice of this invention the promoter region is removed from a cloned gene responsible for male fertility and is replaced with a promoter that only responds to a specific external stimulus. Thus, the gene will not be transcribed except in response to the external stimulus providing the endogenous gene is inactive through mutation or silencing. As long as the gene is not being transcribed, its gene product, which is necessary for completion of pollen development, is not produced. This causes a breakdown in one or more of the biochemical/physiologic pathways of pollen development, which results in sterility. The plant can only become fertile under the specific stimulus that activates the selected promoter.

An example of a responsive promoter system that can be used in the practice of this invention is the glutathione-S-transferase (GST) system in maize. GSTs are a family of enzymes that can detoxify a number of hydrophobic electrophilic compounds that often are used as pre-emergent herbicides (Wiegand, et al., “Messenger RNA Encoding a Glutathione-S-Transferase Responsible for Herbicide Tolerance in Maize is Induced in Response to Safener Treatment”, Plant Molecular Biology 7: 235-243, 1986). It has been discovered that treating maize seed with GSTs increases the tolerance of the maize to the herbicides. Studies have shown that the GSTs are directly involved in causing this enhanced tolerance. This action is primarily mediated through a specific 1.1 kb mRNA transcription product. In short, maize has a naturally occurring quiescent gene already present that can respond to GSTs and that can be induced to produce a gene product. This gene has already been identified and cloned. Thus, the promoter is removed from the GST responsive gene and attached to the fertility gene that previously has had its native promoter removed. This engineered gene is the combination of a promoter that responds to an external chemical stimulus and a gene responsible for successful development of fertile plants.

Another example is a different chemical-inducible gene expression system, using the In2-2 promoter, which is activated by benzenesulfonamide herbicide safeners. De Veylder, L., Van Montagu, M. and Inze, D. “Herbicide Safener-Inducible Gene Expression in Arabidopsis thaliana” Plant Cell Physiol. 38(5) 568-577 (1977).

Inactivation of Native Gene Used in the Present Invention

It will be readily appreciated by those skilled in the art that a wide variety of methods are known to disable the native gene. Homologous recombination is but one of the methods known to those skilled in the art for rendering a native gene inoperative. Thus, when the engineered gene is homologously recombined into the plant, the native gene will be rendered inoperative. A good overview of this general process is provided by Yoder, J. I., and Kmic, Eric, in “Progress Towards Gene Targeting in Plants”, Genetic Engineering, Vol. 13 (Plenum Press, New York, 1991). The authors note “gene targeting can be used to silence or replace the endogenous gene with an engineered allele; thus the phenotype of the altered gene, or its regulatory sequences, can be evaluated in planta.” It is pointed out that genetic recombination takes place through breakage and reunion of DNA and the rejoining mechanism pairs the complimentary DNA sequences. (See, e.g. 271, supra.).

A further discussion of intrachromosomal homologous recombination in plants is discussed at Peterhans, A., et al., “Intrachromosomal Recombination in Plants”, The EMBO Journal, Vol. 9, No. 11, pp. 3437-3445 (1990) and also at Paskowski, J., et al., “Gene Targeting in Plants”, The EMBO Journal, Vol 7, pp. 4021-4026 (1988).

A variety of different means, in addition to these specific examples, would be available to one skilled in the art. Mutations of the native gene can inactivate the gene. A still further example includes backcrossing, using generally accepted plant breeding techniques, to in effect “delete” the native gene. This replaces the native gene with the mutant. Backcrossing is often used in plant breeding to transfer a specific desirable trait from one inbred or source to an inbred that lacks that trait. This can be accomplished for example by first crossing an inbred (A) (recurrent parent) to a donor inbred (non-recurrent parent), which carries the appropriate gene(s) for the trait in question. The progeny of this cross is then mated back to the superior recurrent parent (A) followed by selection in the resultant progeny for the desired trait to be transferred from the non-recurrent parent. After five or more backcross generations with selection for the desired trait, the progeny will be heterozygous for loci controlling the characteristic being transferred, but will be like the superior parent for most or almost all other genes. The last backcross generation would be selfed to give pure breeding progeny for the gene(s) being transferred. A result of any backcrossing method is that the “native” gene is replaced by the desired gene.

A unique method is discussed in a 1991 Science article, reporting on prior work relating to using “transgenic scissors”. This article describes a method in which scientists may remove a marker gene which is attached to a gene having a desired trait in a plant. The “scissor,” according to this method, is an enzyme obtained from a bacterial virus known as “Cre” for control of recombination. Science, p. 1457, Dec. 6, 1991. The enzyme is capable of snipping out any DNA located between a pair of 34-base pair sequences, called lox, for locus of crossing over. This is described in further detail in the patent application filed by Du Pont, and published at WO 91/09957.

Sterility Selection and Fertility Restoration Used in the Present Invention

After the gene is introduced into a plant, the appropriate plant types, the sterile plants, are selected. These plants are sterile because the isolated and cloned fertility gene does not have its native promoter and, therefore, is not producing its gene product that is crucial to successful pollen development. Therefore, the engineered gene acts as a recessive mutant allele of that gene. In normal plant biotechnology, once the desired genotype is identified following transformation and regeneration, the plants are selfed to recover that genotype. However, in the practice of this invention, the desired genotype cannot be selfed at the first generation because it is sterile. To obtain progeny, fertility must be induced by spraying the plants with a compound which induces transcription of the gene by activating the altered promoter. In the case of the GST promoters, the compound is preferably a GST-inducing compound such as N,N-diallyl-2-2-dichloroacetanide. The promoter attached to the fertility gene responds to this chemical and causes the transcription of the gene to begin. Once this occurs, the normal gene product is produced from the gene and some level of fertility is induced.

Once the initial isolation and propagation of the desired genotype is completed, the procedure is more straightforward. Only inbreds that are used as female parents in hybrid crosses are transformed into male sterile variants, where desired only the male parents are transformed into female sterile variants. Once they are transformed, the amount of seed must be increased. This is accomplished by planting in an isolated area (away from other maize pollen) and spraying with a chemical to which the promoter responds. Spraying induces the promoter to start transcription of the gene attached to it. This will produce some degree of fertility.

Any of the female fertility mutants, ba1, ba2, ba3, ba4, lethal ovule 2, barren stalk fastigiated, barren sterile1 and defective pistil, can be readily obtained at the Maize Genetics Cooperation Stock Center, University of Illinois at Urbana/Champaign in Urbana, Ill. (Please see Table 10 for sample sources for the above mutants).

### Example 21

### Male Fertility Genes

Examples of Identifying Genes Critical to Male Fertility Used in the Present Invention

Genetic male sterility results from impacting one of the genes responsible for a specific step in microsporogenesis, the term applied to the entire process of pollen formation. These genes can be collectively referred to as male fertility genes. A male fertility gene may be selected from those genes already known. One such gene is the dam gene derived from Escherichia coli. The dam gene expresses a DNA adenine methylase enzyme in specific plant tissue, which results in the inability of the transformed plants to produce anthers or pollen. Brooks, J. E., et al., “The isolation and characteristics of the Escherichia coli DNA adenine methylase gene” Nucleic Acids Research 11:837-851 (1983). Other genes are the barnase and barstar genes. Mariani, C., et al. demonstrated induction of male sterility in transgenic tobacco and Brassica napus plants by expression of a ribonuclease gene (barnase) from Bacillus amyloliquefaciens in tapetal tissues of anthers using a tapetum-specific promoter. The barstar gene, an intracellular inhibitor of barnase, was used to restore male fertility in barnase-containing lines of B. napus. “A chimaeric ribonuclease-inhibitor gene restores fertility to male sterile plants” Nature 357: 384-387 (June 1992).

There are many steps in the overall pathway where a mutation can lead to male sterility. This seems aptly supported by the frequency of genetic male sterility in maize. New alleles of male sterility mutants are uncovered in materials that range from elite inbreds to unadapted populations.

The procedures for identifying and cloning a male sterile gene are the same as those known in the art to be utilized to clone other genes. The preferred method is transposon (transposable element) tagging because most instances of genetic male sterility in maize are the result of recessive gene mutations. Cloning techniques that require knowledge of the protein sequences of a male sterile gene translation product cannot be used at present because a common gene product of male sterile genes is not yet known.

The procedure for tagging maize genes with transposable elements is known, as reviewed by H. P. Doring, “Tagging Genes With Maize Transposable Elements. An Overview,” Maydica 34 (1989): 73-88, and described in U.S. Pat. No. 4,732,856 to Federoff (“Transposable Elements and Process for Using Same”), the disclosures of which are, as previously noted, incorporated herein in their entirety. An example of a male sterility gene obtained using transposon tagging is found at U.S. Pat. No. 5,478,369.

One of the methods by which this is carried out is by inter-crossing a maize strain carrying active transposable elements and a dominant allele of the target gene involved in microsporogenesis with a normal maize strain that does not carry transposable elements. Specific gene tagging efficiency can be and preferably is enhanced by positioning the transposable element in the proximity of the target gene locus. Progeny from the inter-crosses are selfed and subsequently screened for the most useful mutations. The preferred phenotypes are plants which do not extrude anthers and those which do not produce pollen. Most preferred are phenotypes which do not extrude anthers because this phenotype can easily be screened visually prior to pollination time by gross observation. These male sterile plants represent putative instances in which a transposable element has been excised from its original location and has transposed to a locus bearing a gene which is essential for pollen development. Once the transposable element has transposed to such a locus, the gene is inactivated. It will then behave as a recessive gene and result in male sterility. These mutant plants can be crossed to test stocks for the transposable element to confirm that the element is still present.

Once it has been confirmed that the desired transposable element has transposed into the target gene, genomic clones which hybridize to the transposable element are constructed. The adjacent sequences to the element of the clones are then used as probes in Southern hybridizations with genomic DNA from strains carrying the mutant allele, the revertant allele, and the wild-type allele. The rDNA which reveals the expected differences in size (reflecting the presence or absence of the transposable element) carries the desired modified target gene.

In theory, the frequency with which a particular locus can be targeted with a transposable element usually varies from 10⁻⁵ to 10⁻⁶. In practice, this varies from 10⁻² to 10⁻⁶. Even so, 100,000 maize plants can easily be grown on an area of less than 10 acres. In addition, under certain circumstances the frequency of the element-induced mutations can be increased. For example, the particular transposable element to be used for gene tagging can be linked to the gene to be tagged by the element. For two different transposable element systems, Ac and Spm/En, the transpositions of these elements occurs preferentially to sites on the chromosome where the element was located before the transposition. Alternatively, different transposable elements have different frequencies of mutation induction. For example, the transposable element called Mutator (Mu) is able to induce new mutations at a frequency 30 to 50 times higher than the frequency in control plants. Additionally, the rate of mutation induction can be influenced by the sex of the element carrying parent. While it cannot be predicted which of the reciprocal crosses will give the higher mutation rate, transposon tagging can readily be performed.

At least seven different maize transposable elements have been cloned at this time. These are Ac, Spm/En, Mu, Tz86, Bs1, rDt, and Mpi1. Any of these can be used to clone genes in which a transposable element resides.

One skilled in the art will appreciate this is but one example of means to locate such genes and that other methods are well known.

One collection of mutant genes is already known, and has been described by Albertsen, et al. “Developmental Cytology Genetic Male Sterile Loci in Maize”. Can. J. Genet Cytol. 23: 195-208, (1981), as noted, incorporated herein by reference. These are known as male-sterile (ms) genes. These genes affect development of the pollen only; they have no effect on female organ development. These genes disrupt microsporogenesis at characteristic stages of pollen development, rendering the plant male sterile.

Once the mutant gene from any of the foregoing sources has been cloned, it is used as a probe to clone the wild type allele. This is possible because the mutated gene is very closely similar to the wild type allele, and as such, hybridizes to the wild type allele. Once the normal gene has been identified and cloned, the region of the gene known as a promoter region is identified. This region is involved in the start of transcription of that gene.

Genes which are essential to pollen development can also be identified without intermediate use of mutations by isolating mRNA's that are uniquely present during pollen development and constructing a cDNA that can be used to probe a genomic library for the corresponding gene.

For additional examples of male and female sterility systems and genes, see also, U.S. Pat. Nos. 5,478,369, 5,633,441, 5,824,524, 5,850,014, 5,859,341, 6,265,640, 6,297,426 and 6,815,577, all of which are herein incorporated by reference.

Any of the male fertility mutants, dam, barnase, barstar, silkless1, tasselless and teosinte branched1, previously mentioned above in this application, can be readily obtained at the Maize Genetics Cooperation Stock Center, University of Illinois at Urbana/Champaign in Urbana, Ill. (Please see Table 10 for sample sources for the above mutants).

### Example 22

### Corn Plants Developed with both Female Fertility and Male Fertility Genes Used in the Present Invention

Based on the phenotypes exhibited when a corn plant is developed with a female fertility gene, such as ba3 (D. Nadel, et al., “A new 100% earless recessive trait” Maize Genetic Cooperative Newsletter, Vol. 6 (1990)), and when a corn plant is developed with a male fertility gene such as dam (Brooks, J. E., et al., “The isolation and characteristics of the Escherichia coli DNA adenine methylase gene” Nucleic Acids Research 11:837-851 (1983)), one skilled in the art can predict that a corn plant that is both female-sterile and male-sterile may exhibit a phenotype that is earless with tassels that do not produce viable pollen.

Any of the female fertility genes and male fertility genes, ba1, ba2, ba3, ba4, lethal ovule 2, barren stalk fastigiated, barren sterile 1, defective pistil, dam, barnase, barstar, silkless1, tasselless and teosinte branched1, previously mentioned above in this application, can be readily obtained at the Maize Genetics Cooperation Stock Center, University of Illinois at Urbana/Champaign in Urbana, Ill. (Please see Table 10 for sample sources for the above mutants).

TABLE 10
Maize Genetics Cooperation Stock Center Sources

| Loci | Stock number |
|---|---|
| Barren stalk (ba1) | 318B |
| Barren stalk (ba2) | 218E |
| Barren stalk (ba3) | Ba3926, Ba3964, U740I ba3 |
| Barren stalk (ba4) | Ba4064-1 |
| Lethal ovule 2 (lo2) | 912H lo2 wx1, 912E lo2, 225E telo2L |
| | w3; W3 |
| barren stalk fastigiated (baf1) | 913E |
| Barren sterile1 (bs1) | MBS1038, MBS1527, MBS1538, |
| | MBS165, MBS18, MBS185, MBS1W |
| Defective pistil (dep1) | No stock number - available upon |
| | request |
| dam | Vardaman |
| Barstar | No stock number - available upon |
| | request |
| Silkless1 (sk1) | 208B lg1 gl2 B1 sk1, 208C lg1 gl2 B1 sk1 |
| | v4, 209E lg1 gl2 b1 sk1, 214J sk1, |
| | 427C Ysk1-N844, SK111, SK19 |
| Tasselless (tl) | 127G Tlr1-N1590; 4010G gtl*-N2297; |
| | 4010l gtl*-N2488; 4209A tlr*-N2243 |
| | 4209B tlr*-N2244; 4209D Tlr*-N2444 |
| | 5410B gl*-STL; 5803A shootless*-99- |
| | 677-6; 5806O tls*-Va35 |
| | 5806P tls*-Funk |
| Teosinte branched1 (tb1) | 117D tb1; 117DA tb1-8963; 122C TB1- |
| | Lc; RDTB1 |

### Example 23

### Comparison of Brix Percentage Levels Between Earless and Eared Hybrid Corn 111 Within One Day of Anthesis

Table 11 shows the Brix percentage levels for various nodes for both earless and eared hybrid 111 corn plants taken in Kfar Pines, Israel within one day of anthesis. Hybrid 111 corn is a yellow dent corn. The hybrid corn 111 plants were planted on Mar. 28, 2007 and the Brix readings were taken on Jun. 14, 2007. Column one shows the various nodes, column two shows the Brix percentage levels for earless hybrid corn 111 plants that were grown in a greenhouse, column three shows the Brix percentage levels of earless hybrid corn 111 plants grown in the field and column four shows the Brix percentage levels of eared hybrid corn 111 plants. Note that nodes 1-2 are the nodes closest to ground level, while nodes 11-12 are the highest (farthest from ground level). Data show that there are minor differences in accumulated sugars between the earless and eared corn plants within one day of anthesis, with the differences between the field eared and field earless plants being 1.38% which is equivalent to a 9% total sugars difference. This difference will substantially increase as more sugar resources in the culm of the normal-type corn plants with ears are used in the reproductive cycle.

TABLE 11

| | Brix Percentage of Each Corn Type | | |
|---|---|---|---|
| | | | 111 normal |
| | 111 earless - | 111 earless - | with ears - |
| Nodes | greenhouse | field | field |
| 1-2 | 13.50 | 14.80 | 11.96 |
| 3-4 | 9.50 | 16.56 | 15.68 |
| 5-6 | 13.50 | 16.62 | 16.30 |
| 7-8 | 18.25 | 17.70 | 15.82 |
| 9-10 | 18.25 | 17.44 | 16.86 |
| 11-12 | 12.00 | 16.64 | 14.90 |
| All Nodes | 14.17 | 16.63 | 15.25 |

Table 12 shows the Brix percentage levels for various nodes for both earless and eared hybrid 302 corn plants taken in Kfar Pines, Israel 8 days past anthesis. Hybrid 302 corn is a yellow flint dwarf corn plant. The hybrid corn 302 plants were planted on Mar. 28, 2007 and the Brix readings were taken on Jun. 18, 2007. Column one shows the various nodes, column two shows the Brix percentage levels for earless hybrid corn 302 plants grown in the field and column three shows the Brix percentage levels of eared hybrid corn 302 plants grown in the field. Note that nodes 1-2 are the nodes closest to ground level, while nodes 11-12 are the highest (farthest from ground level). Data show a difference of 2.9% in Brix percentage levels between the eared and earless hybrid 302 corn plants, where the difference will continue to be observed until senescence. When the data from Tables 11 and 12 are taken together, the difference in Brix percentage levels increases because as the grain matures and culm sugar resources are used for all the component parts of the female reproductive system which includes but is not limited to, the shank, cob, silk, embryos-seed, and husks.

TABLE 12

Brix Percentage of Each Corn Type

| | 302 yellow flint dwarf | 302 yellow flint dwarf |
|---|---|---|
| Nodes | earless- field | with ears - field |
| 1-2 | 10.52 | 8.40 |
| 3-4 | 14.28 | 11.20 |
| 5-6 | 15.08 | 11.76 |
| 7-8 | 14.80 | 11.74 |
| 9-10 | 15.06 | 12.04 |
| 11-12 | 15.18 | 12.36 |
| All Nodes | 14.15 | 11.25 |

### Example 24

### Use of Yellow Commercial Corn Hybrid Eared Corn in the Present Invention

Another method of the present invention is using yellow commercial corn hybrid seeds that produced ears. Corn seeds are planted in close proximity to each other. The seeds are then grown to produce hybrid corn plants. Plant material is harvested from the corn plants 21 days before or after anthesis. The juice is then extracted from said corn plants.

What is claimed is:

1. A method of increasing sugar content in the culm comprising the steps of:
a. planting in close proximity, corn seeds which when grown produce an inflorescence-deficient corn plant, a female-sterile corn plant or a corn plant having both female-sterility and male-sterility; and
b. growing said corn seed to produce a corn plant with increased sugar content in the culm.

2. A method of producing juice from corn plants comprising the steps of:
a. planting in close proximity, corn seeds which when grown produce an inflorescence-deficient corn plant, a female-sterile corn plant or a corn plant having both female-sterility and male-sterility;
b. growing said corn seed to produce corn plants;
c. harvesting the plant material from said corn plants; and
d. extracting juice from said plant material to produce juice and residual plant material.

3. A method of producing juice from corn plants comprising the steps of:
a. obtaining corn plant material having a Brix value of at least 10%; and
b. extracting juice from said corn plant material to produce juice and residual plant matter.

4. The method of claim 2, wherein said juice from said corn plants has a sucrose yield of at least about 6.9%.

5. The method of claim 2, wherein said corn plant has a Brix percentage of at least about 10%.

6. The method of claim 2, wherein sucrose and other fermentable sugars are produced from said juice.

7. The method of claim 1, wherein increasing said sugar content is produced from a corn plant having a mutant allele, inherent or transgenetically generated, selected from the group consisting of barren inflorescence1, barren inflorescence2, barren stalk1, barren stalk2, barren stalk3, barren stalk4, barren stalk fastigiate, barren sterile1, defective pistil, lethal ovule2, silkless1, tasselless, teosinte branched1, barnase, barstar and dam.

8. The method of claim 2, wherein said juice is produced from a corn plant having a mutant allele, inherent or transgenetically generated, selected from the group consisting of barren inflorescence1, barren inflorescence2, barren stalk1, barren stalk2, barren stalk3, barren stalk4, barren stalk fastigiate, barren sterile 1, defective pistil, lethal ovule2, silkless1, tasselless, teosinte branched1, barnase, barstar and dam.

9. The plant material of claim 2, wherein fodder having an increased level of digestibility is produced from said plant material.

10. The method of claim 2, further comprising the step of producing molasses from said juice.

11. The method of claim 2, further comprising step e) producing distilled spirits from said juice.

12. The method of claim 2, further comprising step e) producing leaves, tassels and residual bagasse from said residual plant material.

13. The method of claim 2, wherein said corn plant has an earlier maturity date when compared to corn plants grown for grain.

14. The method of claim 2, wherein said plant material is comprised of stalks.

15. The method of claim 2, wherein said juice is removed from said plant material in a field or in a building.

16. The method of claim 2, wherein leaves are removed from said corn plants during the harvesting of said plant material.

17. A method of producing ethanol using juice derived from corn plant material comprising the steps of:
a. growing seed of an inflorescence-deficient corn plant, wherein said corn plant is a female-sterile corn plant or a corn plant having both female-sterility and male-sterililty;
b. harvesting plant material from said corn plant;
c. extracting juice from stalks; and
d. fermenting said juice to produce ethanol.

18. A method of producing ethanol using juice derived from corn plant material comprising the steps of:
a. obtaining corn plant material having a Brix value of at least 10%;
b. extracting juice from said corn plant material to produce juice and residual plant matter; and
c. fermenting said juice to produce ethanol.

19. The method of claim 17, wherein leaves are removed from said stalks.

20. Ethanol produced by the method of claim 17.

21. The method of claim 17, wherein said ethanol is produced from a sterile corn plant having an allele selected from the group consisting of barren1, barren2, barren3, barren-stalk fastigiate, defective pistil mutant, lethal ovule2, teosinte branched1, barnase, barstar and dam.

22. A method for providing fuel needed in the processing of culm juice, wherein said fuel comprises of leaves and bagasse or leaves or bagasse.

23. The method of producing ethanol of claim 17, wherein a higher net energy balance and CO₂ balance is obtained compared to corn grain, sugar beets, and sugarcane.

24. A method for improving sweet sorghum plant yields by using inflorescence-deficient sweet sorghum plants, sorghum plants having female sterility or sweet sorghum plant having both male sterility and female sterility.

25. The method of claim 17, wherein the amount of said ethanol produced per acre day is increased as compared to corn grain, sugar beets or sugarcane.

26. A method of producing juice from corn plants comprising the steps of:
a. planting in close proximity, corn seeds which when grown produce a corn plant capable of producing inflorescences and ears;
b. growing said corn seed to produce corn plants;
c. harvesting the plant material from said corn plants between 21 days prior to anthesis and 21 days after anthesis; and
d. extracting juice from said plant material to produce juice and residual plant material.

27. A method of producing juice from corn plant material derived from corn plants capable of producing inflorescences and ears comprising the steps of:
a. obtaining corn plant material derived from corn plants capable of producing inflorescences and ears and having a Brix value of at least 10%; and
b. extracting juice from said corn plant material to produce juice and residual plant matter.

28. The method of claim 26, wherein leaves are removed from said stalks.

29. The method of claim 26, wherein said juice from said corn plants has a sucrose yield of at least about 6.9%.

30. The method of claim 26, wherein said corn plant has a Brix percentage of at least about 10%.

31. The method of claim 26, wherein sucrose and other fermentable sugars are produced from said juice.

32. The plant material of claim 26, wherein fodder having an increased level of digestibility is produced from said plant material.

33. The method of claim 26, further comprising step e) of producing molasses from said juice.

34. The method of claim 26, further comprising step e) of producing leaves, tassels and residual bagasse from said residual plant material.

35. The method of claim 26, wherein said corn plant has an earlier maturity date relative to corn plants grown for grain.

36. The method of claim 26, wherein said juice is removed from said plant material in a field or in a building.

37. A method of producing ethanol using juice derived from plant material of corn plants comprising the steps of:
a. planting in close proximity, corn seeds which when grown produce a corn plant capable of producing inflorescences and ears;
b. harvesting plant material from said corn plant between 21 days prior to anthesis and 21 days after anthesis;
c. extracting juice from stalks; and
d. fermenting said juice to produce ethanol.

38. The method of claim 34, wherein leaves are removed from said stalks.

39. Ethanol produced by the method of claim 37.

40. Ethanol produced by the method of claim 38.

41. The method of claim 34, wherein the amount of said ethanol produced per acre day is increased as compared to corn grain, sugar beets or sugarcane.

42. A method of producing ethanol using juice derived from corn plant material from corn plants capable of producing inflorescences and ears comprising the steps of comprising the steps of:
a. obtaining corn plant material derived from corn plant material from corn plants capable of producing inflorescences and ears and having a Brix value of at least 10%;
b. extracting juice from said corn plant material to produce juice and residual plant matter; and
c. fermenting said juice to produce ethanol.
